SEQUENCE LISTING (17 sequences)

SEQ ID NO: 1 - DNA, 44 bp, Artificial Sequence
  primer sense
gcgcgatatctagaccaccatggtgctacttgaagactctggat

SEQ ID NO: 2 - DNA, 40 bp, Artificial Sequence
  primer antisense
gcgcgatatcgcggccgctcagtgtggctcagaatggcct

SEQ ID NO: 3 - DNA, 50 bp, Artificial Sequence
  primer sense
gcgcaagcttgtcgacgatatccaccatggtgctacttgaagactctgga

SEQ ID NO: 4 - DNA, 34 bp, Artificial Sequence
  primer antisense
gcgctctagacttcacttcattgagccatatcgg

SEQ ID NO: 5 - DNA, 52 bp, Artificial Sequence
  primer sense
gcgctctagaggatcccccgggctgcaggagcccaaatcttgtgacaaaact

SEQ ID NO: 6 - DNA, 39 bp, Artificial Sequence
  primer antisense
gcgcgcggccgctcatttacccggagacagggagaggct

SEQ ID NO: 7 - DNA, 49 bp, Artificial Sequence
  primer sense
gcgcaagcttgtcgacgatatccaccatgggtggacacagaatggttct

SEQ ID NO: 8 - DNA, 34 bp, Artificial Sequence
  primer antisense
gcgcggatcccttaatttcattgagccatatggg

SEQ ID NO: 9 - DNA, 45 bp, Artificial Sequence
  primer sense
gcgcggatcccccgggctgcaggtgcccagggattgtggttgtaa

SEQ ID NO: 10 - DNA, 47 bp, Artificial Sequence
  primer antisense
gcgcagatctgatatcgcggccgctcatttaccaggagagtgggaga

SEQ ID NO: 11 - DNA, 3696 bp, Artificial Sequence
  DNA sequence encoding human CD163 Fc fusion protein
atggtgctacttgaagactctggatctgctgacttcagaagacattttgtcaacctgagtcccttcaccattactgtggtcttacttctcagtgcctgttttgtcaccagttctcttggaggaacagacaaggagctgaggctagtggatggtgaaaacaagtgtagcgggagagtggaagtgaaagtccaggaggagtggggaacggtgtgtaataatggctggagcatggaagcggtctctgtgatttgtaaccagctgggatgtccaactgctatcaaagcccctggatgggctaattccagtgcaggttctggacgcatttggatggatcatgtttcttgtcgtgggaatgagtcagctctttgggattgcaaacatgatggatggggaaagcatagtaactgtactcaccaacaagatgctggagtgacctgctcagatggatccaatttggaaatgaggctgacgcgtggagggaatatgtgttctggaagaatagagatcaaattccaaggacggtggggaacagtgtgtgatgataacttcaacatagatcatgcatctgtcatttgtagacaacttgaatgtggaagtgctgtcagtttctctggttcatctaattttggagaaggctctggaccaatctggtttgatgatcttatatgcaacggaaatgagtcagctctctggaactgcaaacatcaaggatggggaaagcataactgtgatcatgctgaggatgctggagtgatttgctcaaagggagcagatctgagcctgagactggtagatggagtcactgaatgttcaggaagattagaagtgagattccaaggagaatgggggacaatatgtgatgacggctgggacagttacgatgctgctgtggcatgcaagcaactgggatgtccaactgccgtcacagccattggtcgagttaacgccagtaagggatttggacacatctggcttgacagcgtttcttgccagggacatgaacctgctgtctggcaatgtaaacaccatgaatggggaaagcattattgcaatcacaatgaagatgctggcgtgacatgttctgatggatcagatctggagctaagacttagaggtggaggcagccgctgtgctgggacagttgaggtggagattcagagactgttagggaaggtgtgtgacagaggctggggactgaaagaagctgatgtggtttgcaggcagctgggatgtggatctgcactcaaaacatcttatcaagtgtactccaaaatccaggcaacaaacacatggctgtttctaagtagctgtaacggaaatgaaacttctctttgggactgcaagaactggcaatggggtggacttacctgtgatcactatgaagaagccaaaattacctgctcagcccacagggaacccagactggttggaggggacattccctgttctggacgtgttgaagtgaagcatggtgacacgtggggctccatctgtgattcggacttctctctggaagctgccagcgttctatgcagggaattacagtgtggcacagttgtctctatcctggggggagctcactttggagagggaaatggacagatctgggctgaagaattccagtgtgagggacatgagtcccatctttcactctgcccagtagcaccccgcccagaaggaacttgtagccacagcagggatgttggagtagtctgctcaagatacacagaaattcgcttggtgaatggcaagaccccgtgtgagggcagagtggagctcaaaacgcttggtgcctggggatccctctgtaactctcactgggacatagaagatgcccatgttctttgccagcagcttaaatgtggagttgccctttctaccccaggaggagcacgttttggaaaaggaaatggtcagatctggaggcatatgtttcactgcactgggactgagcagcacatgggagattgtcctgtaactgctctaggtgcttcattatgtccttcagagcaagtggcctctgtaatctgctcaggaaaccagtcccaaacactgtcctcgtgcaattcatcgtctttgggcccaacaaggcctaccattccagaagaaagtgctgtggcctgcatagagagtggtcaacttcgcctggtaaatggaggaggtcgctgtgctgggagagtagagatctatcatgagggctcctggggcaccatctgtgatgacagctgggacctgagtgatgcccacgtggtttgcagacagctgggctgtggagaggccattaatgccactggttctgctcattttggggaaggaacagggcccatctggctggatgagatgaaatgcaatggaaaagaatcccgcatttggcagtgccattcacacggctgggggcagcaaaattgcaggcacaaggaggatgcgggagttatctgctcagaattcatgtctctgagactgaccagtgaagccagcagagaggcctgtgcagggcgtctggaagttttttacaatggagcttggggcactgttggcaagagtagcatgtctgaaaccactgtgggtgtggtgtgcaggcagctgggctgtgcagacaaagggaaaatcaaccctgcatctttagacaaggccatgtccattcccatgtgggtggacaatgttcagtgtccaaaaggacctgacacgctgtggcagtgcccatcatctccatgggagaagagactggccagcccctcggaggagacctggatcacatgtgacaacaagataagacttcaggaaggacccacttcctgttctggacgtgtggagatctggcatggaggttcctgggggacagtgtgtgatgactcttgggacttggacgatgctcaggtggtgtgtcaacaacttggctgtggtccagctttgaaagcattcaaagaagcagagtttggtcaggggactggaccgatatggctcaatgaagtgaagtctagaggatcccccgggctgcaggagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 12 - protein, 1232 aa, Artificial Sequence [sequence omitted]

SEQ ID NO: 13 - DNA, 3693 bp, Artificial Sequence
  DNA sequence encoding murine CD163 fusion protein
atgggtggacacagaatggttcttcttggaggtgctggatctcctggttgtaaaaggtttgtccatctaggtttctttgttgtggctgtgagctcacttctcagtgcctctgctgtcactaacgctcctggagaaatgaagaaggaactgagactggcgggtggtgaaaacaactgtagtgggagagtggaacttaagatccatgacaagtggggcacagtgtgcagtaacggctggagcatgaatgaagtgtccgtggtttgccagcagctgggatgcccaacttctattaaagcccttggatgggctaactccagcgccggctctggatatatctggatggacaaagtttcttgtacagggaatgagtcagctctttgggactgcaaacatgatgggtggggaaagcataactgtacccatgaaaaagatgctggagtgacctgctcagatggatctaatttggagatgagactggtgaacagtgcgggccaccgatgcttaggaagagtagaaataaagttccagggaaagtgggggacggtgtgtgacgacaacttcagcaaagatcacgcttctgtgatttgtaaacagcttggatgtggaagtgccattagtttctctggctcagctaaattgggagctggttctggaccaatctggctcgatgacctggcatgcaatggaaatgagtcagctctctgggactgcaaacaccggggatggggcaagcataactgtgaccatgctgaggatgtcggtgtgatttgcttagagggagcagatctgagcctgagactagtggatggagtgtccagatgttcaggaagattggaagtgagattccaaggagaatgggggaccgtgtgtgatgataactgggatctccgggatgcttctgtggtgtgcaagcaactgggatgtccaactgccatcagtgccattggtcgagttaatgccagtgagggatctggacagatttggcttgacaacatttcatgcgaaggacatgaggcaactctttgggagtgtaaacaccaagagtggggaaagcattactgtcatcatagagaagacgctggtgtgacatgttctgatggagcagatctggaacttagacttgtaggtggaggcagtcgctgtgctggcattgtggaggtggagattcagaagctgactgggaagatgtgtagccgaggctggacactggcagatgcggatgtggtttgcagacagcttggatgtggatctgcgcttcaaacccaggctaagatctactctaaaactggggcaacaaatacgtggctctttcctggatcttgtaatggaaatgaaactactttttggcaatgcaaaaactggcagtggggcggcctttcctgtgataatttcgaagaagccaaagttacctgctcaggccacagggaacccagactggttggaggagaaatcccatgctctggtcgtgtggaagtgaaacacggagacgtgtggggctccgtctgtgattttgacttgtctctggaagctgccagtgtggtgtgcagggaattacaatgtggaacagtcgtctctatcctagggggagcacattttggagaaggaagtggacagatctggggtgaagaattccagtgtagtggggatgagtcccatctttcactatgctcagtggcgcccccgctagacagaacttgtacccacagcagggatgtcagcgtagtctgctcacgatacatagatattcgtctggcaggcggcgagtcctcctgtgagggaagagtggagctcaagacactcggagcctggggtcccctctgcagttctcattgggacatggaagatgctcatgtcttatgtcagcagctgaagtgtggggttgcccaatctattccagaaggagcacattttgggaaaggagctggtcaggtctggagtcacatgttccactgcactggaactgaggaacatataggagattgcctcatgactgctctgggtgcgccgacgtgttccgaaggacaggtggcctctgtcatctgctcaggaaaccaatcccagacactattgccatgtagttcattgtctccagtccaaacaacaagctctacaattccaaaggagagtgaagttccctgcatagcaagtggccagcttcgcttggtaggtggaggtggtcgctgcgctggaagagtggaggtctaccacgagggctcttggggcaccgtctgtgatgacaattgggatatgactgatgccaatgtggtgtgcaagcagctggactgtggcgtggcaattaacgccactggctctgcttacttcggggaaggagcaggagctatctggctagacgaagtcatctgcactgggaaagagtctcatatttggcagtgccattcacatggctggggacgccataactgcaggcacaaagaagatgcaggtgttatctgctccgagttcatgtctctgaggctgaccaacgaagcccacaaagaaaactgcacaggtcgccttgaagtgttttacaatggtacatggggcagtattggcagtagcaatatgtctccaaccactgtgggggtggtgtgccgtcagctgggctgtgcagacaacgggactgtgaaacccataccttcagacaagacaccatccaggcccatgtgggtagatcgtgtgcagtgtccaaaaggagttgacactttgtggcagtgcccctcgtcaccttggaaacagagacaggccagcccctcctcccaggagtcctggatcatctgtgacaacaaaataagactccaggaagggcatacagactgttctggacgtgtggagatctggcacaaaggttcctggggaacagtgtgtgatgactcctgggatcttaatgatgctaaggttgtatgtaagcagttgggctgtggccaagctgtgaaggcactaaaagaagcagcatttggtccaggaactgggcccatatggctcaatgaaattaagggatcccccgggctgcaggtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaa

SEQ ID NO: 14 - protein, 1231 aa, Artificial Sequence
  murine CD 163 fusion protein
MGGHRMVLLGGAGSPGCKRFVHLGFFVVAVSSLLSASAVTNAPGEMKKELRLAGGENNCSGRVELKIHDKWGTVCSNGWSMNEVSVVCQQLGCPTSIKALGWANSSAGSGYIWMDKVSCTGNESALWDCKHDGWGKHNCTHEKDAGVTCSDGSNLEMRLVNSAGHRCLGRVEIKFQGKWGTVCDDNFSKDHASVICKQLGCGSAISFSGSAKLGAGSGPIWLDDLACNGNESALWDCKHRGWGKHNCDHAEDVGVICLEGADLSLRLVDGVSRCSGRLEVRFQGEWGTVCDDNWDLRDASVVCKQLGCPTAISAIGRVNASEGSGQIWLDNISCEGHEATLWECKHQEWGKHYCHHREDAGVTCSDGADLELRLVGGGSRCAGIVEVEIQKLTGKMCSRGWTLADADVVCRQLGCGSALQTQAKIYSKTGATNTWLFPGSCNGNETTFWQCKNWQWGGLSCDNFEEAKVTCSGHREPRLVGGEIPCSGRVEVKHGDVWGSVCDFDLSLEAASVVCRELQCGTVVSILGGAHFGEGSGQIWGEEFQCSGDESHLSLCSVAPPLDRTCTHSRDVSVVCSRYIDIRLAGGESSCEGRVELKTLGAWGPLCSSHWDMEDAHVLCQQLKCGVAQSIPEGAHFGKGAGQVWSHMFHCTGTEEHIGDCLMTALGAPTCSEGQVASVICSGNQSQTLLPCSSLSPVQTTSSTIPKESEVPCIASGQLRLVGGGGRCAGRVEVYHEGSWGTVCDDNWDMTDANVVCKQLDCGVAINATGSAYFGEGAGAIWLDEVICTGKESHIWQCHSHGWGRHNCRHKEDAGVICSEFMSLRLTNEAHKENCTGRLEVFYNGTWGSIGSSNMSPTTVGVVCRQLGCADNGTVKPIPSDKTPSRPMWVDRVQCPKGVDTLWQCPSSPWKQRQASPSSQESWIICDNKIRLQEGHTDCSGRVEIWHKGSWGTVCDDSWDLNDAKVVCKQLGCGQAVKALKEAAFGPGTGPIWLNEIKGSPGLQVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 15 - protein, 22 aa, Artificial Sequence
  N-terminal end of CD163
APGWANSSAGSGRIWMDHVSCR

SEQ ID NO: 16 - protein, 17 aa, Artificial Sequence
  C-terminus end of CD163
EAEFGQGTGPIWLNEVK

SEQ ID NO: 17 - protein, 1116 aa, Homo sapiens
  MISC_FEATURE (1)..(1116): CD163 protein
MVLLEDSGSADFRRHFVNLSPFTITVVLLLSACFVTSSLGGTDKELRLVDGENKCSGRVEVKVQEEWGTVCNNGWSMEAVSVICNQLGCPTAIKAPGWANSSAGSGRIWMDHVSCRGNESALWDCKHDGWGKHSNCTHQQDAGVTCSDGSNLEMRLTRGGNMCSGRIEIKFQGRWGTVCDDNFNIDHASVICRQLECGSAVSFSGSSNFGEGSGPIWFDDLICNGNESALWNCKHQGWGKHNCDHAEDAGVICSKGADLSLRLVDGVTECSGRLEVRFQGEWGTICDDGWDSYDAAVACKQLGCPTAVTAIGRVNASKGFGHIWLDSVSCQGHEPAVWQCKHHEWGKHYCNHNEDAGVTCSDGSDLELRLRGGGSRCAGTVEVEIQRLLGKVCDRGWGLKEADVVCRQLGCGSALKTSYQVYSKIQATNTWLFLSSCNGNETSLWDCKNWQWGGLTCDHYEEAKITCSAHREPRLVGGDIPCSGRVEVKHGDTWGSICDSDFSLEAASVLCRELQCGTVVSILGGAHFGEGNGQIWAEEFQCEGHESHLSLCPVAPRPEGTCSHSRDVGVVCSRYTEIRLVNGKTPCEGRVELKTLGAWGSLCNSHWDIEDAHVLCQQLKCGVALSTPGGARFGKGNGQIWRHMFHCTGTEQHMGDCPVTALGASLCPSEQVASVICSGNQSQTLSSCNSSSLGPTRPTIPEESAVACIESGQLRLVNGGGRCAGRVEIYHEGSWGTICDDSWDLSDAHVVCRQLGCGEAINATGSAHFGEGTGPIWLDEMKCNGKESRIWQCHSHGWGQQNCRHKEDAGVICSEFMSLRLTSEASREACAGRLEVFYNGAWGTVGKSSMSETTVGVVCRQLGCADKGKINPASLDKAMSIPMWVDNVQCPKGPDTLWQCPSSPWEKRLASPSEETWITCDNKIRLQEGPTSCSGRVEIWHGGSWGTVCDDSWDLDDAQVVCQQLGCGPALKAFKEAEFGQGTGPIWLNEVKCKGNESSLWDCPARRWGHSECGHKEDAAVNCTDISVQKTPQKATTGRSSRQSSFIAVGILGVVLLAIFVALFFLTKKRRQRQRLAVSSRGENLVHQIQYREMNSCLNADDLDLMNSSGGHSEPH

(19) United States
(12) Patent Application Publication
(10) Pub. No.: US 2005/0214871 A1
(43) Pub. Date: Sep. 29, 2005

(54) Methods for using the CD163 pathway for modulating an immune response

(76) Inventors: Louis Boon, Amsterdam (NL); Petrus J. Simons, Hillegom (NL); David Speijer, Amsterdam (NL); Ruprecht J.J. van Neerven, Almere (NL)

Correspondence Address: TRASK BRITT, P.O. BOX 2550, SALT LAKE CITY, UT, 84110, US

(21) Appl. No.: 10/996,177

(22) Filed: Nov. 23, 2004

(30) Foreign Application Priority Data
May 27, 2003 (WO) PCT/NL03/00395
May 27, 2002 (EP) 02077071.5

(51) Int. Cl. G01N 33/53; G01N 33/567; C07H 21/04; C12N 15/09; C07K 14/74; C07K 16/28

(52) U.S. Cl. 435/7.2; 435/69.1; 435/320.1; 435/325; 530/350; 536/23.2; 530/388.22

(57) ABSTRACT

The invention relates to the field of immunology, gene therapy, and medicine. More specifically, the invention relates to the identification of a molecule capable of interacting with a soluble and/or cell-bound form of CD163 and, as a result of the interaction, an immune response is either instigated or suppressed in an organism. Furthermore, it relates to the preparation of a pharmaceutical composition including the molecule and/or antagonist I and/or agonist I thereof, and/or an isolated CD163 and/or an antagonist II or agonist II thereof, for the therapeutic or prophylactic treatment of an individual with an immune response disorder, e.g., inflammation, cancer, or infection.

### CROSS-REFERENCE TO RELATED APPLICATION

This application claims priority to International Application No. PCT/NL03/00395, filed May 27, 2003, published in English as PCT International Publication No. WO 03/100419 on Dec. 4, 2003, the contents of which are hereby incorporated herein by this reference in their entirety.

### TECHNICAL FIELD

The invention relates generally to biotechnology, and, more particularly, to the field of immunology, gene therapy, and medicine. More specifically, the invention relates to the identification of a molecule capable of interacting with a cell-bound and/or soluble form of CD163 and, as a result of the interaction, an immune response is either instigated or suppressed in an organism. Furthermore, it relates to the preparation of a pharmaceutical composition comprising the CD163-ligand molecule and/or antagonist I and/or agonist I thereof, and/or an isolated CD163 and/or an antagonist II or agonist II thereof, for the therapeutic or prophylactic treatment of an individual with an immune-related disorder, e.g., inflammation, cancer or infection.

### BACKGROUND

Antigen-presenting cells (“APC”) of the myeloid lineage, such as monocytes, macrophages, and dendritic cells, are key regulators in innate and acquired immune responses. They are capable of capturing and processing antigens and presenting them to T-lymphocytes. The complete activation of T-cells is dependent upon the interaction of co-stimulating molecules on APC (e.g., CD80, CD86, and CD40) with their counterparts on T-cells (CD28, CTLA-4, and CD40L). The cytokines secreted by APC are regulated by the activity of a range of receptors known as pattern-recognition receptors (“PRR”). These receptors are a key feature of the innate immune response because they help discriminate between self and infectious non-self. The PRR include the Toll-like receptors, mannose receptors, and scavenging receptors (Linehan et al. 2000; Imler and Hoffmann 2001). These receptors recognize conserved pathogen-associated molecular patterns, which are shared by large groups of microorganisms, and may also recognize endogenous ligands induced during inflammatory responses. Upon activation via co-stimulatory molecules such as CD40 or via PRR, monocytes, macrophages and dendritic cells are further capable of secreting pro-inflammatory cytokines like, for instance, tumor necrosis factor-alpha (TNF-alpha) and interleukin-1 beta (IL-1 beta) or, depending on the signal, anti-inflammatory cytokines like, for instance, interleukin-10 (IL-10) and transforming growth factor-beta (TGF-beta). Depending on the profile of cytokines produced, immune response generated against a given pathogen are either stimulated or dampened. Further, when activated through PRR, the APC are capable of initiating type 1 (inflammatory) versus type 2 (antibody-mediated) immune responses (Kopp and Medzhitov 1999; Imler and Hoffmann 2001; Re and Strominger 2001), largely dependent on the amount of IL-12 they produce.

One of these pattern-recognition receptors, human CD163 (also recognized as M130 or RM3/1), was identified as a membrane protein that is expressed on macrophages and on monocytes (Morganelli et al. 1988; Zwadlo et al. 1987; Law et al. 1993; Pulford et al. 1992; Sulahian et al. 2000). cDNAs encoding murine (Schaer et al. 2001, 2002), as well as human CD163 (Law et al. 1993, Högger et al. 1998; Ritter et al. 1999), have been identified and have shown that CD163 is a member of the cysteine-rich scavenger receptor (SRCR) protein superfamily type B. The CD163 gene encodes a 130-155 kDa transmembrane protein. In addition, several isoforms have been described that differ in the length of the cytoplasmic region (45, 84 and 89 amino acids, respectively), suggesting that these isoforms may have differential signaling and function (Högger et al. 1998; Law et al. 1993; Ritter et al. 1999).

CD163 can be removed from the cell membrane, for example, upon treatment in vitro using the potent inflammatory mediators phorbol 12-mysristate-13-acetate (PMA) or lipopolysaccharide (LPS) (Droste et al. 1999; Hintz et al. 2002). This CD163 down-regulation is the result of protease-mediated shedding of the receptor, rather than endocytosis of CD163, resulting in a soluble protein named sCD163 (sCD163).

An ELISA to detect sCD163 in human serum has been published and reagents are available (Sulahian et al. 2001). Using this assay, it was shown that sCD163 is present in serum from healthy volunteers and, therefore, also exists in vivo. In addition, plasma levels of CD163 were shown to be up-regulated after cardiac surgery (Sulahian et al. 2001; and WO 01/73435, and U.S. 20010041177), as well as after infusion of LPS into healthy human volunteers (WO 01/73435 and U.S. 20010041177). Both these situations resemble acute inflammation, and TNF-alpha, IL-6 and cortisol are also detectable in plasma. Levels of sCD163 are also increased in arthritis (Matsushita et al. 2002), Gaucher disease (Möller et al. 2002-b), and in the serum of patients with myelomonocytic leukemias and infections (Möller 2002-a). This indicates that sCD163 acts as an acute phase protein during an inflammatory response. A potential anti-inflammatory effect of sCD163 was demonstrated (Högger et al. 2001; Frings et al. 2002). These experiments showed that sCD163 inhibited phorbol-ester-induced human T-cell proliferation in vitro and thus may attenuate immune responses in vivo.

CD163 has recently been shown to be involved in hemoglobin (Hb) metabolism (Kristiansen et al. 2001). Efficient removal of free Hb is essential for health because of the oxidative and toxic properties of the iron-containing haem in Hb. The elevated expression of CD163 on tissue macrophages is, therefore, in line with its physiological important function in Hb catabolism of macrophages. Free Hb released into the serum is complexed by the acute phase protein haptoglobin and the complexes of haptoglobin and Hb are scavenged by CD163 that only recognizes the complex and not the components alone (Kristiansen et al. 2001). Upon binding to CD163, the haptoglobin-hemoglobin complexes are endocytosed by the macrophages, whereupon the haem present in Hb is converted into bilirubin and iron. This was confirmed by Schaer et al. (Schaer et al. 2002), who disclose the notion that this action of CD163 may be a direct anti-inflammatory action of corticosteroids. This finding is in line with the finding that haem oxygenase-1 is induced by hemoglobin as well as by IL-10, and mediates the anti-inflammatory effect of IL-10 and hemoglobin (Otterbein et al. 1995; Lee and Chau 2002). These data link macrophage haem metabolism to anti-inflammatory responses and also suggest an important role for CD163 in anti-inflammatory responses. Further, immunohistochemical analysis has shown that CD163-positive macrophages accumulate in inflammatory sites during the healing phase of both acute and chronic inflammation, suggesting a role for CD163-positive macrophages with the healing phase of the inflammatory response (Zwadlo et al. 1987).

Freshly isolated human monocytes have a low-level expression of CD163. Upon in vitro culture of such monocytes in the presence of macrophage colony-stimulating factor (M-CSF) the monocytes differentiate into macrophages. This is accompanied by up-regulation of CD163 mRNA and protein (Buechler et al. 2000). When monocytes are cultured in the presence of granulocyte/macrophage colony-stimulating factor (GM-CSF) and IL-4, factors that induce differentiation into dendritic cells, CD163 expression is down-regulated (Buechler et al. 2000). Interestingly, dendritic cells are the central cells involved in initiating immune responses in vivo, and therefore, the rationale of the low-expression levels of CD163 on these cells may be related to the strong immunostimulatory capacity of these cells.

Although (gluco)corticosteroids are the most widely used immunosuppressive and anti-inflammatory agent in current clinical medicine, their pharmacological activities involved in modulation of the immune system are poorly understood. Unlike the role of CD163 in Hb catabolism, scavenger receptor cysteine-rich (SRCR) proteins are typically associated with immune function and are, therefore, expressed on cells from the immune system. Interestingly, the expression of CD163 on monocytes and macrophages is up-regulated in vitro by anti-inflammatory mediators such as IL-10 and (gluco)corticosteroids. Further, in vivo administration of corticosteroids to human volunteers resulted in an increase both in antigen density and the number of cells that express CD163, six hours after administration (Zwadlo-Klarwasser et al. 1990). On the other hand, pro-inflammatory mediators like LPS, interferon-gamma (IFN-gamma) and TNF-alpha suppress expression of CD163 on these cells.

Even though many of the findings described above suggest an anti-inflammatory role for membrane CD163, stimulation of macrophages by cross-linking CD163 with the mAb (mAb) EDHU-1 has been reported to induce the production of the pro-inflammatory cytokines IL-1 beta, IL-6 and GM-CSF (Ritter et al. 2001; van den Heuvel et al. 1999). CD163 signal transmission upon stimulation depends on a protein tyrosine kinase (PTK) activity resulting in calcium mobilization and inositol triphosphate generation. Since the cytoplasmic domains of CD163 and its isoforms contain no known PTK motif, the molecular mechanisms by which CD163 signal transduction is mediated are not understood. In addition to the PTK-dependent signal, another non-characterized signal pathway has been described that is independent of PTK activity. More recently, interaction with the regulatory subunit of casein kinase II and protein kinase C with the cytoplasmic tail of CD163 on macrophages have been observed (Ritter et al. 2001).

### DISCLOSURE OF THE INVENTION

Surprisingly, we disclose herein that ligation of CD163 by another CD163-specific mAb, RM3/1, down-regulates the production of the pro-inflammatory cytokine TNF-alpha in response to LPS, while up-regulating the anti-inflammatory cytokine IL-10. This, therefore, proves that immune responses will be dampened when CD163 binds to its ligand(s). We further demonstrate herein that apart from its role in Hb catabolism, CD163 (i.e., membrane-bound (mCD163) and/or soluble (sCD163) form) has immunoregulatory properties mediated in part through binding/interaction with a novel ligand (referred to herein as CD163-ligand) besides haptoglobin-hemoglobin complexes.
sCD163 has an immunomodulatory effect that can be mediated in part by binding either to a CD163-ligand present on the cell surface of antigen-specific T-lymphocytes or to its soluble form (sCD163-ligand). Preferably, such an immunomodulatory effect comprises stimulation of an immune response.

In one embodiment, the invention thus provides an agonist of CD163/CD163-ligand signaling in the form of antibody RM3/1. The invention thus provides the use of antibody RM3/1 or a functionally related molecule for the dampening of an immune response in an individual. A functionally related molecule is a part of RM3/1 that is capable of recognizing the same epitope as RM3/1. Suitable parts are those that retain a variable part of antibody RM3/1, such as FAB fragments or single-chain variable fragments thereof. A functional equivalent of RM3/1 is also a different antibody comprising the same epitope-recognition capabilities as RM3/1. Such antibodies may be selected from CD163-binding antibodies by competition assays with RM3/1. Those which compete with binding to CD163 with RM3/1 are likely to have the same effect as RM3/1. It is, of course, not excluded that among the other CD163-binding antibodies, there are those that have similar effects as RM3/1. Such antibodies are also part of the invention. An antibody of the invention is preferably a human or humanized antibody. At least part of the variable domain of RM3/1 or functionally related molecule may be grafted on a human antibody backbone to generate a humanized version of RM3/1 or the related molecule.

Apoptosis is defined as genetically programmed cell death, which provides a counterbalance to mitosis in the regulation of tissue growth and homeostasis. Apoptosis is characterized by cell shrinkage, retention of organelles and nuclear chromatin condensation, which is accompanied by nuclear fragmentation. In many cell lineages, apoptosis is featured by blebbing of plasma membranes, which leads to detachment of membrane-enclosed apoptotic bodies. Apoptotic bodies can contain organelles and even nuclear fragments (Millset al. 1999; Willingham et al. 1999; Savill et al. 2002).

It is generally accepted that macrophages are the most important cells in the body, which recognize and remove apoptotic cells and their related products. However, non-professional phagocytes, like dendritic cells, fibroblasts, hepatocytes, epithelial and endothelial cells, are also described to mediate clearance of apoptotic cells (Fadok et al. 2001).

Many surface receptors on macrophages are believed to contribute to the initial recognition and binding of apoptotic cells. These include lectins and integrins, the class A and class B scavenger receptors (CD36, SRA, CD68, LOX-1), complement receptors, the phosphathidylserine (PtdSer) receptor and the endotoxin receptor CD14 (Fadok et al. 2001; Savill et al. 2002). After recognition and binding of apoptotic cells, rapid engulfment of dying cells by macrophages is promoted. There is growing evidence from many studies that the net effect of all these interactions results in anti-inflammatory and immunosuppressive responses from macrophages (Fadok et al. 1998; McDonald et al. 1999; Voll et al. 1997; Byrne et al. 2002). This is exemplified by the induction of anti-inflammatory factors, like IL-10 and TGF-beta, and inhibition of pro-inflammatory factors, like IL-1 beta and TNF-alpha, from LPS-stimulated macrophages, which have been previously exposed to apoptotic cells. In addition, the antitumor activity of macrophages is seriously impaired after exposure of macrophages to apoptotic tumor cells (Reiter et al. 1999).

Primary necrosis is defined as accidental or murderous cell death resulting from, e.g., exposure to toxins, hypoxia or temperature extremes. Necrosis is characterized by cell swelling, leading to bursting of cells, and consequently the release of free organelles and cellular contents (Willingham et al. 1999; Savill et al. 2002). Secondary or post-apoptotic necrosis is the eventual disintegration of apoptotic cells when the clearance of apoptotic cells is impaired (Savill et al. 2002; Nauta et al. 2003).

In contrast to apoptotic cells, primary necrotic cells are a toxic and pro-inflammatory activator of macrophages, due to the release of cellular contents (Fadok et al. 2001; Savill et al. 2002; Reiter et al. 1999). For this reason, it postulated that autoimmunity could be induced in situations of defective apoptotic cell removal, which results, as stated above, in secondary necrotic cells (Savill et al. 2002; Nauta et al. 2003).

In summary, the rapid uptake of intact apoptotic cells by macrophages and the coinciding anti-inflammatory/immunosuppressive local micro-environment are of great in vivo importance to prevent damaging of neighboring cells and triggering of unwanted inflammatory (auto)immune responses. On the other hand, apoptotic tumor cells could induce anti-inflammatory/immunosuppressive responses as a mechanism to escape immune surveillance.

The novel ligand for CD163 disclosed herein interacts with membrane CD163, induces the production of IL-10, which, in turn, induces the production of haem oxygenase 1 and up-regulates the expression of CD163, which enables the CD163-expressing cells to efficiently remove Hp-Hb complexes from the circulation. In addition, the resulting induction of haem oxygenase-I will exert a direct anti-inflammatory effect. Thus, the use of a CD163 ligand for inducing haem-oxygenase-I in a subject is provided.

For example, CD163 binds to apoptosis-related proteins on the cell surface of apoptotic cells, such as proteins belonging to the histone family. As described above, apoptotic cells need to be removed from the body to prevent them from leaking their contents and inducing necrosis. It is known that the binding of apoptotic cells by macrophages leads to an inhibition of the production of pro-inflammatory cytokines such as TNF-alpha and to the induction of IL-10 and other anti-inflammatory factors (Fadok et al. 1998; McDonald et al. 1999; Voll et al. 1997; Byrne et al. 2002). We disclose that interaction of CD163 on the macrophage/monocyte with these apoptosis-related proteins (e.g., proteins belonging to the histone family) may be a key event in the inhibition of the production of pro-inflammatory cytokines such as TNF-alpha and to the induction of IL-10 and other anti-inflammatory factors during the uptake and degradation of the apoptotic cells. The invention thus further provides a method to interfere with apoptosis-related events in a subject, comprising providing the subject with a means for modulating CD163/CD163-ligand signaling in the subject. The means may comprise an agonist of CD163/CD163-ligand signaling and/or an antagonist thereof. Such means is preferably a soluble CD163, a soluble CD163-ligand, a CD163-specific antibody, a CD163-ligand-specific antibody or a histone or functional part, derivative and/or analogue of such a molecule. The invention further provides a method for detecting apoptotic cells comprising providing the cells with a CD163 molecule or functional part, derivative and/or analogue thereof. The invention further provides a method for detecting apoptotic cells comprising providing the cells with a histone-specific antibody or functional part, derivative and/or analogue thereof. The invention further provides a method for detecting a histone, particularly histone H2A, H2B and H4, characterized in that a CD163 molecule, or a functional part, derivative and/or analogue thereof is used.

It was demonstrated (Wu et al. 2002) that nucleosomal histones become separated from DNA within a few hours during apoptosis and are detectable in cell lysates prepared by using a non-ionic detergent. Here, we disclose that apoptotic cells express molecules (within hours) that interact with CD163 from macrophages. These molecules were identified as nucleosomal histones: H2A, H2B and H4. We disclose that the interaction of CD163 on macrophages with histones on apoptotic cells play a role in the induction of anti-inflammatory and immunosuppressive responses observed during the subsequent phagocytosis and elimination of apoptotic cells.

Inflammation is a major hallmark of a wide range of diseases such as autoimmune diseases, inflammatory diseases, organ rejection, and infectious diseases. Inflammation is characterized by an influx of cells of the immune system, the release of cytokines and other inflammatory mediators such as histamine, leukotriens and prostaglandins, resulting in fever and tissue destruction. Inflammatory processes include: the local reactions and resulting morphologic changes; the destruction or removal of the injurious material; and the responses that lead to repair and healing. The typical signs of inflammation are redness, heat or warmth, swelling, pain, and occasionally inhibited or lost function. In the present invention, it was found that by manipulating the CD163/CD163-ligand signaling pathway, it is possible to modulate an immune response in a subject. The immune response plays a role in many different diseases. Those vary from typical immune system diseases such as auto-immune diseases and infections, etc., to diseases where the immune system has a more hidden role such as leptin metabolism. The immune system is affected for example in disturbed fat metabolism, where changes in leptin levels directly influence immune function.

Pharmaceutical agents used to modulate inflammation in a host should ideally be endogenous substances, such as therapeutical proteins. These therapeutical proteins should not be recognized as foreign agents (i.e., no neutralizing antibodies will be formed, no nephrotoxicity is expected from endogenous proteins and no complexes are expected to be formed due to an antibody response against the therapeutical protein). Although many pharmaceutical compounds are currently used to treat inflammatory diseases, there is a need for more effective compounds (immunosuppressive molecules) with lowered toxicity profiles.

Currently used pharmacological therapies in immune-related diseases in many cases only provide temporal relief In addition, drugs used to combat inflammation are not very selective, targeting non-inflammatory cells as well as inflammatory cells and often have moderate to serious side effects after chronic treatment, especially in children. Many patients become resistant to the drugs (e.g., glucocorticoid) used and high doses are associated with unpleasant side effects. Hence, there is a strong need for safer, more selective and more efficacious therapies which display a long-term clinical benefit to patients suffering from immune-related disorders.

Inflammation often accompanies, and is a response to, infection or other injury; however, chronic and autoimmune inflammation represent undesirable pathological conditions in which infection is not typically present. In these latter diseases, one wishes to inhibit inflammation.

In infection and cancer on the other hand, one wishes to stimulate the inflammatory response to generate more aggressive immune responses to the infectious agent and/or the tumor cell. Cancer and infectious diseases are significant health problems throughout the world. Although advances have been made in detection and therapy of these diseases, no vaccine or other universally successful method for prevention or treatment is currently available.

Infection is defined by the invasion and multiplication of foreign microorganisms such as viruses, bacteria, fungi including yeast, and parasites, in the body. Infections are generally harmful to the host, resulting in local cellular injury. A local infection may persist and spread by extension to become an acute, subacute or chronic clinical infection or disease state.

Many of the frequently occurring infectious diseases of today are caused by bacteria, which have gained resistance to drugs previously effective in treating diseases caused by these bacteria, or by viruses. Only a small number of antiviral drugs are currently available for treatment of virus infections. A complication to the development of such drugs is that mutant strains of virus that are resistant to currently available antiviral drugs develop readily.

As with the antiviral agents, the development of anticancer agents for treating cancer effectively has also been problematic. Cancer therapy currently relies on a combination of early diagnosis and aggressive treatment, which may include radiotherapy, chemotherapy or hormone therapy. However, the toxicity of such treatments limits the use of presently available anticancer agents. The high mortality rate for many cancers indicates that improvements are needed in cancer prevention and treatment.

Many tumors escape surveillance by our immune system. In cancer patients, there is clearly a quantitative and/or qualitative defect in the immune system's specific mechanisms to eliminate tumor cells. It is clear that there is still a great need to find ways to generate and/or enhance protective anti-tumor responses involving cellular and humoral immunity. In fact, tumor cells can produce anti-inflammatory cytokines such as IL-10 and TGF-beta, and certain tissues in mammals with cancer of the immune and circulatory systems express significantly reduced levels of the pro-inflammatory cytokine TNF-alpha.

### BRIEF DESCRIPTION OF THE DRAWINGS

FIG. 1 shows purification of human-soluble CD163. Human monocytes were isolated as described in Example 1. After a four-day culture in the presence of 200 nM dexamethasone, shedding of CD163 was induced by treating the cells with 50 nM PMA for one hour in serum-free PBS. The supernatants were collected and sCD163 was purified from these supernatants by conventional column chromatography methods as described in detail in Example 1. Lane 1 is an Mw marker; Lane 2 is the supernatant before purification; and Lane 3 is purified human-soluble CD163.

FIG. 2 depicts detection of a ligand to CD163 present on apoptotic T-cells. Apoptosis was induced in the human T-cell line Jurkat E6-1 by treating the cells with anti-CD95 antibody (clone CH11; 20 ng/ml). The cells were collected at several time points, treated with sCD163-biotin for 30 minutes, and binding of sCD163-biotin was detected using streptavidin-phycoerythrin, followed by detection in a flow cytometer. The closed curves represent cells not treated with sCD163-biotin and the open curves represent cells treated with sCD163-biotin.

FIG. 3 represents induction of low-Mw proteins after induction of apoptosis in Jurkat E6-1 T-cells. Apoptosis was induced in the human T-cell line Jurkat E6-1 by treating the cells for four hours with anti-CD95 antibody (clone CH11; 20 ng/ml). As a control, untreated cells were used. The cells were lysed in the non-ionic detergent Nonidet P40 (NP40) and run on an SDS-PAGE gel. Upon induction of apoptosis (Lane 2), three distinct bands at approximately 10 to 20 kDa were induced that were not present in cells that were not treated with anti-CD95 antibody (Lane 1).

FIG. 4 shows detection of CD163-ligand molecules in a western blot of apoptotic Jurkat E6-1 cells. Apoptosis was induced in the human T-cell line Jurkat E6-1 by treating the cells for four hours with anti-CD95 antibody (clone CH11; 20 ng/ml). The cells were lysed in the non-ionic detergent Nonidet P40 (NP40) and run on an SDS-PAGE gel. After transfer of the proteins to nitrocellulose, the blots were treated with sCD163-biotin (Lanes 2) or left untreated (Lanes 3). Lanes 1 represent an Mw marker. The binding of sCD163-biotin was detected using streptavidin-HRP to detect the biotin moiety (FIG. 4A) as well as using anti-CD163-HRP to detect the sCD163 (FIG. 4B).

FIG. 5 is the N- and C-terminus of human sCD163. The figure is a schematic representation of the membrane-bound CD163 protein as encoded by the full length CD163 gene, indicating the N- and C-terminus of sCD163. To determine the N- and C-terminus of sCD163, peptide mass fingerprinting was performed on purified sCD163 as described in detail in Example 17. The most C-terminal peptide detected was EAEFGQGTGPIWLNEVK (SEQ ID NO:______) and the most N-terminal peptide detected was APGWANSSAGSGRIWMDHVSCR (SEQ ID NO:______).

FIG. 6 is the human CD163-Fe construct. A schematic representation of the construct is shown in FIG. 6A. FIG. 6B shows a Western blot of the human CD163-Fc construct as described in Example 18. After 10% SDS-PAGE, reduced proteins were transferred to nitrocellulose. Human CD163-Fc was revealed by incubation with anti-human IgG HRP followed by staining with 4-chloro-1-naphtol. Lane 1 control Sf9 cell-lysate; Lane 2 human CD163-Fc-transfected Sf9 cell-lysate; Lane 3 molecular weight markers.

FIG. 7 represents the murine CD163-Fc construct. A schematic representation of the construct is shown in FIG. 7A. FIG. 7B shows a western blot of the murine CD163-Fc construct expressed as described in Example 18. After 10% SDS-PAGE, reduced proteins were transferred to nitrocellulose. Murine CD163-Fc was revealed by incubation with anti-murine IgG HRP followed by staining with 4-chloro-1-naphtol. Lane 1 molecular weight markers; Lane 2 murine CD163-Fe-transfected Sf9 cell-lysate; Lane 3 control Sf9 cell-lysate.

FIG. 8 shows that cathepsins B and D remove CD163 from the cell surface of CD163-expressing cells. To study the effect of isolated proteinases on cell surface CD163, monocytes were isolated from huffy coats as described in Example 1 and cultured for four days in the presence of dexamethasone to induce high levels of CD163-expression. The monocytes/macrophages were collected and incubated in the presence of PMA or without cathepsin B, cathepsin C, cathepsin D, cathepsin G, or elastase and incubated for one hour at 37° C. in PBS, 0.1% BSA. Expression of CD163, Mannose receptor, CD14, CD36, CD86 and HLA-DR was assessed by FACS analysis. Clearly, cathepsin B and cathepsin G were able to remove CD163 from the cell membrane, but not other membrane proteins such as CD 14, CD36, or HLA-DR.

FIG. 9 depicts the induction of IL-10 and inhibition of TNF-alpha by anti-CD163 antibody RM3/1. Purified peripheral blood monocytes were cultured with 200 nM dexamethasone for four days. Subsequently, these matured macrophage subpopulations (1×10⁶/ml) were stimulated with various concentrations of LPS in the absence (open bars) or in the presence of anti-CD163 antibody RM3/1 (black bars; 10 microgram IgG1/ml). After 24 hours LPS treatment, TNF-alpha and IL-10 levels were measured in culture supernatants. Results are presented as mean +/−standard deviation from two separate donors. The figure shows that IL-10 production is enhanced, while TNF-alpha production is decreased when the cells are treated with RM3/1 in the presence of LPS.

### DETAILED DESCRIPTION OF THE INVENTION

As described above, immune-related disorders may be due to either immune heightening, such as in the case of auto-immune diseases, or immune dampening, such as in the case of cancer and infection. The present invention, through the identification of a ligand for CD163 (membrane-bound and/or soluble/secreted/shed form), provides an efficacious method to modulate undesirable immune responses through CD163 or its ligand, termed CD163-ligand.

The invention provides a method for identifying a molecule with immune-modulatory activity capable of interacting with CD163 comprising providing CD163 or a functional part, derivative and/or analogue thereof, and under suitable conditions, detecting a molecule capable of interacting with CD163 and determining whether the molecule is capable of modulating an immune response. “CD163” as used herein refers to the membrane-bound and/or its soluble (i.e., secreted/shed) form. For instance, CD163 is a receptor or functional fragment thereof present on cells of myeloid lineage (monocytes, macrophages, dendritic cells) and/or cells of lymphoid lineage and its soluble form (sCD163) is the shed product of this receptor. A “molecule” as used herein can be any substance be it nucleic acid, amino acid, a carbohydrate or a lipid comprising moiety (or combinations thereof), or any other moiety that can interact with CD163 and, as a result of that interaction, an immune response is modulated (i.e., instigated or suppressed). It is understood that the molecule with immune-modulatory activity can be cell/membrane-bound and/or soluble. It is also understood that the molecule can block the active site of CD163 and in doing so can modulate an immune response. The “active site” as used herein is the binding/adherence site on CD163 for a particular substance (e.g., a functional site such as a receptor-binding cavity). It should be noted that CD163-ligand does not necessarily bind to the same region of the CD163 molecule as haptoglobin-hemoglobin complexes do. In a preferred embodiment of a method of the invention, the molecule with immune-modulatory activity is present on cells of lymphoid lineage (e.g., T-cells, B-cells) or endothelium and comprises a CD163-ligand. In an even more preferred embodiment of the invention, the expression of CD163-ligand on the cell membrane is enhanced after induction of apoptosis, and the CD163-ligand is a member of the histone family, more particularly comprising histones H2A, H2B and H4.

For example, a CD163-ligand can bind to membrane-bound CD163 (mCD163), thus inhibiting the production of pro-inflammatory cytokines such as TNF-alpha, and inducing the production of anti-inflammatory cytokines such as IL-10, by CD163-bearing cells, serving to suppress an immune response. On the other hand, a CD163-ligand (e.g., sCD163-ligand) can be used to neutralize CD163 (e.g., sCD163) in situations where sCD163 is increased. This can, at least in part, reduce the immunostimulatory effects of sCD163 and serve to suppress an immune response.

An immune response is a physiological response of an organism to agents (e.g., infectious agents, proteins, tumor cells, etc.) that pose a threat to that organism. For example, an immune response can involve activation and production of factors by leucocytes comprising B-lymphocytes and T-lymphocytes, NK cells, granulocytes, monocytes, macrophages and dendritic cells. Two types of immune responses are well recognized, namely, the innate immune response and the adaptive immune response. An adaptive immune response is highly antigen-specific and can generate long-lived immune memory. Cells involved in the adaptive immune response (henceforth called antigen-specific immune response), such as B-lymphocytes and T-lymphocytes, recognize their antigens through highly specific cell surface receptors. Innate immune responses are responses involving granulocytes and monocytes, macrophages and dendritic cells and NK cells. These cells recognize frequently encountered antigens with germline-encoded PRR, or the constant region of antibodies, thus providing a first line of defense before the acquired immune system is able to mount a response. PRR, when they bind their ligand, transmit signals into the immune cell which can lead to the release of biological mediators that can instruct the adaptive (acquired), as well as innate, arm of the immune response.

“Modulation” as used herein can refer to up-regulation or down-regulation of an immune response, for example, by activation and/or suppression of gene(s) which are essentially capable of initiation, progression, suppression, and/or repression of an immune response and/or symptoms of the immune response. Modulation can be mediated by positive regulation (i.e., up-regulation) or negative regulation (i.e., down-regulation) of gene transcription and/or by the modification of a gene and/or gene product (e.g., post-translational modification). A functional part of CD163 is defined as a part of CD163 which has the same properties (e.g., CD163-ligand-binding property) as CD163 in kind, but not necessarily in amount. A functional derivative of CD163 is defined as CD163 which has been altered such that the ligand-binding properties of the altered CD163 are essentially the same in kind, but not necessarily in amount. Suitable derivatives can be generated through using codon degeneracy, for example, by conservative amino acid substitution. A functional analogue of CD163 is a homologue and/or functional equivalent of CD163 which can be derived from a different species and/or generated synthetically.

In a preferred embodiment of a method according to the invention, CD163 comprises sCD163. sCD163 is a secreted or shed form of the membrane-bound CD163. In another preferred embodiment of a method of the invention, the molecule with immune-modulatory activity comprises a proteinaceous molecule, functional derivative, functional fragment and/or analogue thereof. A “proteinaceous molecule” as used herein can be any amino acid comprising moiety. In one embodiment, the proteinaceous molecule comprises a CD163-ligand. A CD163-ligand can be a proteinaceous molecule encoded by a nucleic acid of a cell. A functional part of the CD163-ligand is defined as a part of a CD163-ligand which has the same immunomodulatory properties as a CD163-ligand in kind, but not necessarily in amount. By “immunomodulatory properties” is meant the capability to induce or inhibit an immune response in a host T-cell. A “functional derivative of a CD163-ligand” is defined as a CD163-ligand which has been altered such that the properties (e.g., immunomodulatory properties) of altered CD163-ligand are essentially the same in kind, but not necessarily in amount. Suitable derivatives can be generated through using codon degeneracy, for example, by conservative amino acid substitution. A functional analogue of the CD163-ligand is a homologue and/or functional derivative (i.e., functional equivalent) of the CD163-ligand which can be derived from a different species and/or generated synthetically. As used herein, the term “functional equivalent” means that the amino acid of a proteinaceous molecule, according to the invention, can be modified by means of one or more substitutions, deletions, or additions, the net effect of which does not result in a functional dissimilarity in kind, not necessarily in amount, between the amino acid of a proteinaceous molecule according to the invention and the modified form.

In a preferred embodiment, the invention provides an isolated, recombinant and/or synthetic molecule obtainable by a method according to the invention. In one aspect, the invention provides a substantially isolated or purified CD163-ligand or a recombinant (i.e., a modified form generated through genetic engineering approaches) or a synthetic (i.e., artificially generated as opposed to naturally occurring, for example, by chemical synthesis) form thereof having substantially similar immunomodulatory activity. As used herein, the term “substantially isolated or purified” refers to a molecule according to the invention that is removed from its natural environment, isolated or separated, and is essentially free from components with which it is naturally associated. In terms of the invention, the phrase “substantially similar immunomodulatory activity” means that the natural, recombinant or synthetic CD163-ligand polypeptide, according to the invention, or any oligopeptide thereof, is similarly immunologically active in kind, not necessarily in amount; that is, it is capable of inducing a specific immune response in a mammal or a cell. Preferably, the molecule is of mammalian or avian origin. It is understood that the CD163-ligand can be cell/membrane-bound (e.g., a cell surface protein or receptor), for example, on cells of lymphoid lineage, endothelial cells and/or myeloid lineage, or soluble (e.g., freely circulating), and can be involved in apoptosis. For instance, the soluble form of the CD163-ligand can arise as a result of shedding of the membrane-bound form or be produced as a soluble molecule. Preferably, the CD163-ligand-soluble form (sCD163-ligand) comprises at least part of the extracellular and/or cytoplasmic domain, more preferably, the CD163-binding domain of a cell/membrane-bound CD163-ligand (mCD163-ligand). An APC can be any cell (e.g., macrophages, endothelium, dendritic cells, langerhans cells of the skin, etc.) which carries on its surface antigenic peptides bound to major histocompatibility complex (e.g., MHC Class I or Class II) molecules and presents the antigen in this context to cells of the immune system. It is also understood that a molecule of the present invention can inhibit or induce signal transduction into APC. Preferably, binding of the molecule to CD163 results in inhibition of the production of pro-inflammatory cytokines as TNF-alpha while increasing the production of anti-inflammatory cytokines such as IL-10 by APC (e.g., macrophages).

In another preferred embodiment, the invention provides a CD163-ligand molecule obtainable by a method according to the invention. Preferably, the CD163-ligand molecule comprises a protein belonging to the histone family (e.g., H2A, H2B or H4) or functional fragment thereof. For instance, the CD163-ligand can be a protein belonging to the histone family or functional part thereof induced on cells of lymphoid lineage and/or endothelial cells and/or cells of myeloid lineage (monocytes, macrophages, dendritic cells) after induction of apoptosis and its soluble form (sCD163-ligand) is the shed and/or soluble product of this protein, which may be capable of freely circulating in body fluids.

In another aspect, the invention provides a CD163-ligand molecule according to the invention coupled to a moiety. For instance, a CD163-ligand molecule or functional fragment thereof, such as a proteinaceous molecule of the invention, can be linked to a second or subsequent moiety to form a fusion protein. In terms of the invention, the moiety can serve to confer additional properties to the proteinaceous molecule (e.g., increased longevity and stability, improved intracellular targeting) or to improve its biological activity (i.e., immunomodulatory activity and/or pharmacokinetics). Suitable moieties can include a molecule comprising at least part of an immunoglobulin chain (e.g., a constant region of the chain), a molecule with immunoregulatory activity like a cytokine (e.g., IL-10, IL-12, granulocyte colony-stimulating factor (GCSF), IFN-gamma, etc.), a toxic moiety, and a protection molecule like polyethylene glycol (PEG). It is understood that by using a nucleic acid encoding a molecule or a fragment thereof of the present invention, any combination of fusion protein(s) can also be generated using recombinant techniques known in the art. For example, a fusion protein of CD163-ligand (e.g., sCD163-ligand) coupled to the constant region of an immunoglobulin (e.g., sCD163-ligand-Fc), can be used to neutralize CD163 (e.g., sCD163) and/or to cross-link membrane-bound CD163 to inhibit the production of pro-inflammatory cytokines and enhance the production of anti-inflammatory cytokines by CD163-expressing cells. This can, at least in part, reduce the immunostimulatory effects of sCD163 and serve to stimulate an immune response. Such a fusion protein has an advantage over a monovalent sCD163-ligand alone in that it has a longer life in circulation and, as a result of its bivalency, may have an additive effect on inhibiting immune responses.

In another aspect, the invention provides use of a molecule according to the invention to modulate an immune response. The immune response can comprise an innate and/or an antigen-specific (adaptive) immune response. Preferably, the immune response comprises an antigen-specific (adaptive/acquired immune) response.

In inflammatory diseases, such as autoimmune diseases, allergy, asthma, transplant rejection, the activation of the immune system is either unwanted or excessive. These diseases are highly specific for the antigens that are recognized by the immune system, such as auto-antigens (autoimmune diseases), allo-antigens (transplant rejection), allergens (allergy) or infectious agents (certain infections).

In yet another aspect, the invention provides use of a CD163-ligand molecule and/or functional derivative, functional fragment, and/or analogue thereof according to the invention for modulating an immune response wherein modulation comprises inhibition of an immune response. As a way of illustration but not as a way of limitation, by modulating the activity of CD163 and/or a CD163-ligand of the present invention (such as sCD163-ligand), one is capable of suppressing an immune response in auto-immune disease like rheumatoid arthritis, diabetes, multiple sclerosis, systemic lupus erythematosus, psoriasis, autoimmune thyroiditis, inflammatory diseases, transplantation diseases, infectious diseases (i.e., septic shock), etc. This can be achieved in a number of ways.

The capability to interfere with CD163/CD163-ligand signaling is one of the hallmarks of the present invention. The invention provides several means for manipulating this signaling. The invention provides both agonist and antagonists of signaling. Agonists are capable of, at least in part, mimicking CD163/CD163-ligand signaling (either in the CD163 positive cell or in the CD163-ligand positive cell). The effect of such agonists is thus to dampen an immune response. Antagonists are capable of, at least in part, preventing CD163/CD163-ligand signaling and are thus capable of stimulating an immune response when compared to the situation in the absence of the antagonist. Agonists can be divided into two groups. Agonists of group I are capable of binding CD163-ligand, whereas agonists of group II are capable of binding CD163. Antagonists are likewise dividable into two groups. Group I is capable of binding to CD163 ligand, whereas antagonists of group II are capable of binding to CD163.

One way to inhibit an immune response is to stimulate the production of IL-10 and inhibit the production of pro-inflammatory cytokines. For example, by providing an excess of sCD163-ligand and/or an excess of sCD163-ligand coupled to a moiety as mentioned previously, and/or an excess of an agonist II and/or an agonist II coupled to a moiety, one can directly stimulate the production of IL-10 and inhibit the production of pro-inflammatory cytokines by CD163-bearing cells.

Another way to inhibit an immune response is by neutralizing sCD163-induced immune stimulation in situations in which sCD163 levels are increased. For example, by providing an excess of sCD163-ligand and/or an excess of sCD163-ligand coupled to a moiety as mentioned previously, and/or an excess of an antagonist of CD163 (termed antagonist II) and/or an excess of an antagonist II coupled to a moiety, one can neutralize the stimulating effects of sCD163 in situations where sCD163 levels are increased. The sCD163-ligand can engage the sCD163 shed from cell membranes under disease conditions and this can effectively restore the ability of membrane-bound CD163 to interact with the cell-bound CD163-ligand on T-cells or other CD163-ligand-bearing cells, thus dampening the immune response.

It is understood that a CD163-ligand molecule with immune-modulatory activity according to the invention (e.g., sCD163-ligand) can induce or prevent signal transduction into CD163-bearing cells, like macrophages. Interaction of a membrane-bound and/or sCD163-ligand molecule of the invention with membrane-bound CD163 modulates activation and cytokine production of a cell bearing CD163 (e.g., an APC, like a macrophage) by enhancing the production of anti-inflammatory cytokines (like, for instance, IL-10) while inhibiting the production of pro-inflammatory cytokines (like, for instance, TNF-alpha). This may or may not depend on the valency of the binding. The molecule of the invention (e.g., sCD163-ligand) or an agonist 11 to CD163 of the invention is particularly suitable for the inhibition of immune responses in inflammatory diseases.

A molecule of the invention (e.g., sCD163-ligand) or an antagonist 11 to CD163 of the invention is also particularly suitable for the inhibition of immune responses in diseases in which serum sCD163 levels are increased by neutralizing the stimulatory effect of sCD163.

In another aspect, the invention provides an antagonist of a CD163-ligand molecule according to the invention. The antagonist, further referred to as “antagonist I,” is a substance that, at least in part, tends to nullify the action of a molecule of the invention, whether the molecule is membrane-bound or soluble. Preferably, antagonist I completely blocks the binding of CD163-ligand to CD163. Preferably, antagonist I is an antibody or a functional part, derivative and/or analogue thereof. A functional part of an antibody is defined as a part which has the same kind of binding properties in kind, not necessarily in amount (e.g., a Fab fragment). A functional derivative of an antibody is defined as an antibody which has been altered such that the binding properties of antibody are essentially the same in kind, not necessarily in amount. A derivative can be provided in many ways, for instance, through conservative amino acid substitution. A non-limiting example of a suitable derivative is a single-chain antibody. A non-limiting example of a suitable analogue is a synthetic antibody selected from a recombinant antibody library. In a preferred embodiment, an antibody of the invention is a mAb or a functional part, derivative and/or analogue thereof. A mAb of the invention can be generated by standard immunization and cell fusion techniques or by combinatorial library approaches. It is also understood that antagonist I of the present invention can comprise a chimeric antibody, a humanized antibody or a fully human antibody. It is also understood that antagonist I can comprise an antibody with a neutralizing and/or blocking function.

The invention further provides an antagonist I coupled to a moiety. In terms of the invention, the moiety can serve to confer additional properties to antagonist I (e.g., increased longevity and stability, improved intracellular targeting) or to improve its biological activity (i.e., immunomodulatory activity and/or pharmacokinetics). Suitable moieties can include a molecule comprising at least part of an immunoglobulin chain (e.g., a constant region of the chain), a molecule with immunoregulatory activity like a cytokine (e.g., IL-10, IL-12, GCSF, IFN-gamma, etc.), a toxic moiety, and a protection molecule like polyethylene glycol (PEG). It is understood that by using a nucleic acid encoding an antagonist I, or a fragment thereof, any combination of fusion protein(s) can also be generated using recombinant techniques known in the art.

The invention as disclosed herein thus provides a method(s) using antibodies of an isolated and/or recombinant and/or synthetic proteinaceous molecule according to the invention to induce or prevent signaling through CD163 and/or CD163-ligand. An antibody of the present invention can bind to mCD163-ligand and/or sCD163-ligand and can either block binding of the CD163-ligand to CD163 or can induce or enhance signaling through the CD163-ligand. Thus, an antibody of the present invention can have immunomodulatory effects, either by blocking of signaling through CD163 or by directly affecting signaling through CD163-ligand. It is understood that an antibody of the present invention can bind to mCD163-ligand and/or sCD163-ligand and have no immunomodulatory activity (i.e., will not influence signaling through the CD163-ligand). It is also understood that an antibody of the present invention can induce the mCD163-ligand to be internalized, shed from the membrane, or secreted. Moreover, an antibody of the present invention can be coupled to an effector molecule (e.g., a cytokine) to increase the immunomodulatory capacity of the antibody. Additionally, an antibody of the present invention can be coupled to a toxic moiety, allowing the reduction or a depletion of cells expressing the CD163-ligand.

In another aspect the invention provides use of an antagonist I to modulate an immune response. An antagonist I CD163-ligand-specific antibody of the present invention can bind the CD163-ligand on CD163-ligand-bearing cells and can inhibit the binding of CD163-ligand to CD163, thus neutralizing the CD163-ligand-induced induction of anti-inflammatory cytokine production in CD163-bearing cells. Furthermore, an antagonist I CD163-ligand-specific antibody of the present invention can induce or enhance the signaling that is induced by signal transduction through the CD163-ligand or prevent the interaction of CD163-ligand with CD163 or sCD163. This can counteract (i.e., at least in part nullify) the CD163-ligand-mediated dampening of immune responses. Also, an antagonist I CD163-ligand-specific antibody of the present invention can induce shedding or endocytosis of CD163-ligand, thus decreasing CD163-ligand expression on CD163-ligand-bearing cells, hence serving to induce an immune response.

Therefore an antagonist I can be used to modulate an immune response, preferably in a CD163-related pathway, wherein modulation comprises augmentation (i.e., amplification) of an immune response. For example, an antagonist I can be used to stimulate immune responses in cancer, in infections, and in other diseases in which no proper immune response is mounted. With an antagonist I (e.g., CD163-ligand-specific antibody) of the present invention, an antagonist II (e.g., sCD163-specific antibody) of the invention, and/or a molecule according to the invention (e.g., sCD163 or sCD163-Fc), it is now, for instance, possible to efficiently block the immune-dampening effect of interaction of CD163-ligand with membrane CD163. This serves to boost an organism's immune response against tumor cells or where there is an insufficient response against infectious pathogens. In this case, the membrane-bound form of the CD163-ligand (mCD163-ligand) is prevented from binding to CD163 or sCD163 and is, therefore, able to prevent the production of anti-inflammatory cytokines such as IL-10, while enabling the production of pro-inflammatory cytokines such as TNF-alpha by CD163-expressing cells such as macrophages, resulting in fewer persistent tumors and/or infections. Furthermore, antagonist I antibodies of a CD163-ligand may mediate direct activation of CD163-ligand-expressing immune cells like T- and/or B-cells, resulting in fewer persistent tumors or infections.

The invention also provides an agonist of a CD163-ligand molecule according to the invention. The agonist, further referred to as agonist I, is a substance that tends to, at least in part, induce a negative signal through CD163-ligand into the cell-expressing CD163-ligand, resulting in modulation of an immune response. Preferably, the agonist I is an antibody or a functional part, derivative and/or analogue thereof. A functional part of an antibody is defined as a part which has the same kind of binding properties in kind, not necessarily in amount (e.g., a FAB fragment). A functional derivative of an antibody is defined as an antibody which has been altered such that the binding properties of the antibody are essentially the same in kind, not necessarily in amount. A derivative can be provided in many ways, for instance, through conservative amino acid substitution. A non-limiting example of a suitable derivative is a single-chain antibody. A non-limiting example of a suitable analogue is a synthetic antibody selected from a recombinant antibody library. In a preferred embodiment, an antibody of the invention is a mAb or a functional part, derivative and/or analogue thereof. A mAb of the invention can be generated by standard immunization and cell fusion techniques or by combinatorial library approaches. It is also understood that the agonist I can comprise a chimeric antibody, a humanized antibody or a fully human antibody.

An agonist I can bind a molecule of the invention, for instance, mimicking the effects of binding of sCD163 to a CD163-ligand molecule of the invention. It is understood that an agonist I can bind with a higher avidity to a molecule of the invention. This can give rise to at least a similar or even a superior signaling through a CD163-ligand (e.g., membrane-bound). Such a signal will result in suppression of an immune response. Preferably, an agonist I does not completely inhibit the binding of CD163-ligand to CD163 (e.g., mCD163, sCD163).

In another aspect, the invention provides an agonist I coupled to a moiety. For instance, linked to a second or subsequent moiety to form a fusion protein. In terms of the invention, the moiety can serve to confer additional properties to the agonist I (e.g., increased longevity and stability, improved intracellular targeting) or to improve its biological activity (i.e., immunomodulatory activity and/or pharmacokinetics). Suitable moieties can include a molecule comprising at least part of an immunoglobulin chain (e.g., a constant region of the chain), a molecule with immunoregulatory activity like a cytokine (e.g., IL-10, IL-12, GCSF, IFN-gamma, etc.), a toxic moiety, and a protection molecule like polyethylene glycol (PEG). It is understood that by using a nucleic acid encoding an agonist I, or a fragment thereof, any combination of fusion protein(s) can also be generated using recombinant techniques known in the art.

Additionally, the invention provides use of an agonist I to modulate an immune response. Preferably, modulation comprises suppression of an immune response. It is understood that an agonist I, through interacting with a CD163-ligand, can serve to inhibit an immune response, for instance, an antigen-specific (adaptive) immune response. An agonist I can, by itself or in association with CD163, deliver a signal (e.g., inhibitory signal) through the CD163-ligand on CD163-ligand-bearing cells and this can inactivate the immune system.

An agonist I according to the invention is particularly efficacious for the prophylactic or therapeutic treatment of immunological diseases, such as, for instance, autoimmune diseases, inflammatory diseases, transplant rejection, or infectious diseases. Such an agonist I can, at least in part, prevent activation of CD163-ligand-bearing cells, such as T-cells or other cells of the immune system, resulting in dampening of an immune response. Preferably, the immune response comprises an antigen-specific (adaptive immune) response. Furthermore, such agonist I may, at least in part, prevent or overcome endocytosis or shedding and/or secretion of CD163-ligand from CD163-ligand-bearing cells, thereby increasing the net amount of CD163-ligand present on CD163-ligand-bearing cells.

The invention further provides an isolated and/or recombinant and/or synthetic CD163, a functional part, derivative and/or analogue thereof. Preferably, CD163 comprises a sCD163. In a preferred embodiment of the invention, sCD163 comprises a C-terminus and/or N-terminus identical to the natural or PMA-induced shed CD163 molecule. Preferably, the N-terminal end comprises APGWANSSAGSGRIWMDHVSCR (SEQ ID NO:______) and the C-terminus comprises EAEFGQGTGPIWLNEVK (SEQ ID NO:______). The invention provides a substantially isolated or purified CD163 or a recombinant (i.e., a modified form generated through genetic engineering approaches) or a synthetic (i.e., artificially generated as opposed to naturally occurring, for example, by chemical synthesis) form thereof having substantially similar immunomodulatory activity. As used herein, the term “substantially isolated or purified” refers to CD163 according to the invention removed from its natural environment, isolated or separated, and is essentially free from components with which it is naturally associated. In terms of the invention, the phrase “substantially similar immunomodulatory activity” means that the natural, recombinant or synthetic CD163 polypeptide according to the invention, or any oligopeptide thereof, is similarly immunologically active in kind, not necessarily in amount. A functional part of CD163 is defined as a part of CD163 which has the same properties as CD163 in kind, but not necessarily in amount. A functional derivative of CD163 is defined as CD163 which has been altered such that the CD163-ligand-binding properties of the altered CD163 are essentially the same in kind, but not necessarily in amount. Suitable derivatives can be generated through using codon degeneracy, for example, by conservative amino acid substitution. A functional analogue of CD163 is a homologue and/or functional equivalent of CD163 which may be derived from a different species and/or generated synthetically.

The invention further provides an isolated CD163 coupled to a moiety. For instance, an isolated CD163, a functional part, derivative and/or analogue thereof according to the invention can be linked to a second or subsequent moiety to form a fusion protein. In terms of the invention, the moiety can serve to confer additional properties to the isolated CD163 molecule (e.g., increased longevity and stability, improved intracellular targeting) or to improve its biological activity (i.e., immunomodulatory activity and/or pharmacokinetics). Suitable moieties can include a molecule comprising at least part of an immunoglobulin chain (e.g., a constant region of the chain), a molecule with immunoregulatory activity like a cytokine (e.g., IL-10, IL-12, GCSF, IFN-gamma, etc.), a toxic moiety, and a protection molecule like polyethylene glycol (PEG). In a preferred embodiment of the present invention, the moiety comprises a constant region of an immunoglobulin. CD163 is preferably linked to the constant region of an immunoglobulin by the C-terminus EAEFGQGTGPIWLNEVK (SEQ ID NO:______) to prevent proteolytic cleavage of the CD163-Fc molecule.

In another aspect, the invention provides an antagonist of CD163. An antagonist, further referred to as antagonist II of CD163, can, at least in part, neutralize or block CD163 (e.g., sCD163). Preferably, an antagonist II can completely block the interaction of CD163 with CD163-ligand. By neutralizing CD163, the binding/interaction of a molecule of the invention (e.g., CD163-ligand) with CD163 (e.g., sCD163) is at least in part reduced. Neutralizing CD163 can serve to counteract the dampening effects of CD163 on immune responses, like T-cell responses. For instance, an antagonist II can serve to augment an immune response through neutralization of sCD163, by preventing the interaction of CD163 with the CD163-ligand. Preferably, antagonist II is an antibody or functional part, derivative and/or analogue thereof. It is also understood that the antagonist can comprise an antibody with a neutralizing and/or blocking function.

The invention also provides an antagonist II coupled to a moiety. In terms of the invention, the moiety can serve to confer additional properties to the antagonist II of CD163 (e.g., increased longevity and stability, improved intracellular targeting) or to improve its biological activity (i.e., immunomodulatory activity and/or pharmacokinetics). Suitable moieties can include a molecule comprising at least part of an immunoglobulin chain (e.g., a constant region of the chain), a molecule with immunoregulatory activity like a cytokine (e.g., IL-10, IL-12, GCSF, IFN-gamma, etc.), a toxic moiety, and a protection molecule like polyethylene glycol (PEG). For example, antagonist II can comprise an antibody which can be coupled to a toxic moiety, allowing the reduction or a depletion of cells expressing CD163. In the same manner, a toxic moiety can also be coupled to sCD163 or a fragment thereof, to reduce or eliminate its target cells.

The invention further provides an agonist of CD163, further referred to as agonist II. Preferably, agonist II is an antibody or functional part, derivative and/or analogue thereof. It is understood that agonist II can serve to inhibit an immune in a similar manner to a CD163-ligand molecule of the invention, through its ability to stimulate the production of anti-inflammatory cytokines such as IL-10 while inhibiting the production of pro-inflammatory cytokines such as TNF-alpha in CD163-expressing cells. The invention further provides an agonist II coupled to a moiety. In terms of the invention, the moiety can serve to confer additional properties to agonist II of CD163 (e.g., increased longevity and stability, improved intracellular targeting) or to improve its biological activity (i.e., immunomodulatory activity and/or pharmacokinetics). Suitable moieties can include a molecule comprising at least part of an immunoglobulin chain (e.g., a constant region of the chain), a molecule with immunoregulatory activity like a cytokine (e.g., IL-10, IL-12, GCSF, IFN-gamma, etc.), a toxic moiety, and a protection molecule like polyethylene glycol (PEG).

Furthermore, the invention provides a method to detect the presence of a CD163-ligand molecule according to the invention in a sample comprising contacting the sample with a binding molecule for the CD163-ligand molecule according to the invention to form a complex, further comprising detecting the complex in the sample. For example, the binding molecule is any entity, be it a nucleic acid, an amino acid, a carbohydrate or a lipid-comprising moiety (or combinations thereof), that can bind a molecule of the invention. Preferably, the entity is coupled/linked to a moiety which enables the detection of a molecule of the invention in a sample.

Preferably, the binding molecule comprises a CD163-ligand-binding antibody according to the invention. More preferably, the antibody is an antagonist I and/or agonist I according to the invention. For example, antagonist I or agonist I can have a moiety attached, which can be, for example, recognized by a detection/determining substance (e.g., a label). For instance, a visually detectable or direct label (e.g., radioactive label, enzyme label, fluorescent label, chemiluminescent label, bioluminescent label, gold label, etc.). Examples of commonly used enzyme labels are horseradish peroxidase, alkaline phosphatase and beta-galactosidase, etc. Also preferred is that the binding molecule comprises sCD163 and/or mCD163.

In yet another aspect, the invention provides a method to determine the binding activity of a CD163-ligand molecule according to the invention in a sample comprising detecting the presence of a molecule using a method according to the invention, further determining the levels (i.e., amount) of binding molecule-molecule (i.e., complex) in the sample. It is, therefore, an object of the present invention to monitor an immune response comprising detecting the presence of and/or monitoring the binding activity of a CD163-ligand molecule (e.g., sCD163-ligand) in a sample. Screening technologies are known in the art, for example, proteomic technologies.

The invention provides a method for the production of a diagnostic kit comprising a method to detect the presence of a CD163-ligand molecule according to the invention in a sample and/or to determine the binding activity of a CD163-ligand molecule according to the invention in a sample. A suitable basis for a diagnostic kit are known in the art.

In one aspect, the invention provides a nucleic acid, functional part, functional derivative and/or analogue thereof encoding a CD163-ligand molecule according to the invention. A functional part of a nucleic acid of the invention is a part of the nucleic acid whose encoded product is capable of modulating an immune response, preferably in a CD163-related pathway, in an organism. A functional derivative of a nucleic acid of the invention is any nucleic acid produced from or related to the nucleic acid, which retains the same properties as the nucleic acid in kind, not necessarily in amount. An analogue of a nucleic acid of the invention can be, for example, an allelic variant.

In another aspect, the invention provides a nucleic acid functional part, functional derivative and/or analogue thereof encoding an antagonist I of a CD163-ligand molecule according to the invention. In another aspect, the invention provides a nucleic acid, functional part, functional derivative and/or analogue thereof encoding an agonist I of a CD163-ligand molecule according to the invention. In yet another aspect, the invention provides a nucleic acid encoding an isolated CD163 according to the invention. In yet another aspect, the invention provides a nucleic acid encoding an antagonist II or an agonist II of an isolated CD163 according to the invention.

In yet another aspect, the invention provides a vector comprising a nucleic acid according to the invention. Suitable vectors are known to one of skill in the art, for example, plasmid vectors, viral vectors etc. In another aspect, the invention provides a cell comprising a vector according to the invention, preferably, a mammalian or avian cell. Furthermore, the invention provides a gene delivery vehicle comprising a vector according to the invention. A gene delivery vehicle as used herein is any vehicle that can deliver a nucleic acid of the invention to an organism, for the purpose of modulating an immune response in an organism. The invention also provides the use of a gene delivery vehicle of the invention for the preparation of a medicament.

The invention provides a method for the production of a CD163-ligand molecule according to the invention, or an antagonist I of a molecule according to the invention, or an agonist I of a molecule according to the invention, or CD163 according to the invention, or an antagonist II of CD163 according to the invention, or an agonist II of CD163 according to the invention in an organism comprising inserting into the genome of that organism one or more copies of a nucleic acid according to the invention. An organism in the context of the present invention can be, for example, a micro-organism (e.g., archaea, bacteria, cyanobacteria, micro-algae, fungi, yeast, viruses, protozoa, rotifers, nematodes, micro-crustaceans, micro-mollusks, micro-shellfish, micro-insects, etc.), a plant, a non-human animal, and a plant or animal cell (e.g., artificial cell, cell culture or protoplast, etc.).

In yet another aspect, the invention provides use of an isolated CD163 according to the invention and/or an antagonist II of CD163 according to the invention to modulate an immune response. For instance, CD163 according to the invention and/or an antagonist II can be linked to a second or subsequent moiety to form a fusion protein. In terms of the invention, the moiety can serve to confer additional properties to the CD163 molecule (e.g., increased longevity and stability, improved intracellular targeting) or to improve its biological activity (i.e., immunomodulatory activity and/or pharmacokinetics). Suitable moieties can include a molecule comprising at least part of an immunoglobulin chain (e.g., a constant region of the chain), a molecule with immunoregulatory activity like a cytokine (e.g., IL-10, IL-12, GCSF, TNF-alpha, IFN-gamma, etc.), a toxic moiety, and a protection molecule like polyethylene glycol (PEG).

For example, the use of a fusion protein of CD163 (e.g., sCD163) coupled to the constant region of an immunoglobulin (e.g., CD163-Fc) can have several advantages over unmodified CD163. For instance, a CD163-Fc has an increased half-life in circulation. Additionally, a CD163-Fc can be generated as a monovalent (i.e., one CD163 coupled to the constant region of an immunoglobulin chain) molecule, or more preferred as a bivalent (i.e., one or two CD163 moieties coupled to the constant region of an immunoglobulin chain) molecule. A bivalent CD163-Fc molecule is likely to block a molecule of the invention (e.g., CD163-ligand) with a higher avidity than a monovalent sCD163. For instance, an isolated CD163 according to the invention and/or an isolated CD163 coupled to a moiety can, upon binding to a CD163-ligand molecule of the invention, preferably a membrane-bound molecule, at least in part, block the binding to mCD163 with CD163-ligand, resulting in inducing/stimulating/augmenting of an immune response. CD163 and/or CD163 coupled to a moiety can, for instance, bind monovalently, bivalently and/or multivalently to a molecule according to the invention (i.e., CD163-ligand). Preferably, the immune response comprises an immune response to a cancer cell or an infectious agent, such as a virus, a parasite, a fungus, or a bacterium. In the case of an immune response which comprises an immune response to a cancer cell or an infectious agent such as a virus, a parasite, a fungus, or a bacterium, CD163 and/or CD163 coupled to a moiety can be used to suppress unwanted and uncontrolled tumor growth and also eradicate infections with pathogens through its stimulating effect on immune responses.

An antagonist II of CD163 can, at least in part, prevent CD163 from binding/interacting with a CD163-ligand molecule of the invention, thereby preventing the delivery of a signal though CD163 resulting in the production of anti-inflammatory cytokines. For instance, antagonist II of CD163 can be a blocking antibody, or a functional derivative thereof. An antagonist II of CD163 of the present invention is particularly suitable for inducing/stimulating/augmenting an immune response in an organism, (e.g., mammal), in which the immune system is not efficiently stimulated, as is the case in cancer and infection.

It should be noted that substances that can remove CD163 from the cell surface, either by down-regulating CD163 gene expression or by active removal of the molecule from the surface, have similar immunological effects as antagonists II and are thus equivalents of antagonist II. Such equivalents are also part of the invention, as well as method and uses.

Further, in diseases with increased serum levels of sCD163, an antagonist II according to the invention is capable of binding to sCD163 and of, at least in part, neutralizing the immune-stimulating effect of sCD163. With an antagonist II, it is now, for instance, possible to efficiently decrease unwanted high levels of circulating sCD163 in patients suffering from inflammatory diseases without harmful side-effects.

In another aspect, the invention provides use of sCD163 and/or sCD163 coupled to a moiety and/or an antagonist II and/or an antagonist II coupled to a moiety to modulate an immune response. The immune response can comprise an innate and/or an antigen-specific (adaptive) immune response. Preferably, the immune response comprises an antigen-specific (adaptive) immune response. In the case of an immune response which comprises an insufficient immune response as seen in cancer and infections, sCD163 and/or sCD163 coupled to a moiety and/or an antagonist II and/or an antagonist II coupled to a moiety can be used to stimulate immune responses to effectively remove tumor cells and eradicate infection.

In the case of diseases with increased serum levels of sCD163, excessive immune responses can be dampened by an excess of antagonist II and/or an antagonist II coupled to a moiety that efficiently neutralize sCD163.

The invention further provides an agonist II that is capable of at least interacting (e.g., binding) with mCD163, modulating the activation of a cell bearing CD163, resulting in the enhanced production of anti-inflammatory cytokines like IL-10, and decreased production of pro-inflammatory cytokines, such as TNF-alpha. For instance, agonist II of CD163 can be a blocking antibody or a functional derivative thereof. Preferably, agonist II is the antibody RM3/1, an antibody that has the same functional effect on CD163-expressing cells, or an antibody that recognizes the same epitope as the RM3/1 antibody.

Thus, by modulating the activation of CD163-bearing cells, an immune response is dampened. Preferably an agonist II does not completely block interaction of CD163 with CD163-ligand. It should be noted, however, that an agonist II may completely block the interaction of CD163 with CD163-ligand, but is discriminated from an antagonist II on the basis of its functional effect on CD163-expressing cells (i.e., stimulating production of IL-10 while inhibiting production of TNF-alpha).

In another aspect, the invention provides use of an agonist II of CD163 and/or an agonist II coupled to a moiety to modulate an immune response. The immune response can comprise an innate and/or an antigen-specific (adaptive) immune response. Preferably, the immune response comprises an antigen-specific (adaptive) immune response. Even more preferably, modulation of an immune response with an agonist II comprises dampening/suppression/inhibition of an immune response. With an agonist II, it is now, for instance, possible to treat diseases characterized by excessive immune activation, such as, for instance, autoimmune diseases, inflammatory diseases, or transplant rejection.

In another aspect the invention provides a pharmaceutical composition comprising a CD163-ligand molecule according to the invention, an antagonist I of a CD163-ligand molecule according to the invention, an agonist I of a CD163-ligand molecule according to the invention, an isolated CD163 according to the invention, an antagonist II of an isolated CD163, an agonist II of an isolated CD163, a cell according to the invention, and/or a gene delivery vehicle according to the invention. Suitable basis for pharmaceutical compositions are known in the art. Pharmaceutically acceptable carriers are well known in the art and include, but are not limited to, saline, buffered saline, dextrose, water, glycerol, sterile isotonic aqueous buffer, and combinations thereof. One example of such an acceptable carrier is a physiologically balanced culture medium containing one or more stabilizing agents such as stabilized, hydrolyzed proteins, lactose, etc. The carrier is preferably sterile.

In yet another aspect, the invention provides a pharmaceutical composition according to the invention for augmenting or suppressing an immune response. Depending on the use (i.e., to enhance or suppress an immune response), a therapeutic or a prophylactic effective amount of a second substance (i.e., anti-inflammatory/immunosuppressive substance like cyclosporin A, FK506, sulfasalazine, antihistamines, bronchodilators, leukotrien inhibitors, (gluco)-corticosteroids, anti-TNF-alpha antibodies, anti-CD40 antibodies, CpGs, or immunostimulatory substances like type I interferon, GCSF, IFN-gamma or other cytokines, adjuvants to induce inflammation) can be added to the pharmaceutical composition. The pharmaceutical composition can also be used in combination with various cancer treatments, treatments for infectious diseases (e.g., antibacterial, antifungal or antiviral substances), or vaccines comprising tumor antigens and/or antigens derived from infectious agents.

In another aspect, the invention provides use of a CD163-ligand molecule according to the invention, an antagonist I of a CD163-ligand molecule according to the invention, an agonist I of a CD163-ligand molecule according to the invention, an isolated CD163 according to the invention, an antagonist II of an isolated CD163 according to the invention, an agonist II of an isolated CD163 according to the invention, a cell according to the invention, and/or a gene delivery vehicle according to the invention in the preparation of a pharmaceutical composition for the therapeutic or prophylactic treatment of a disease, treatable by modulating an immune response.

On one hand, an isolated CD163 according to the invention, an isolated CD163 of the invention coupled to a moiety (e.g., the constant region of an immunoglobulin (e.g., sCD163-Fc)), an antagonist I of CD163-ligand, an antagonist I of CD163-ligand coupled to a moiety, an antagonist II of CD163, and/or an antagonist II of CD163 coupled to a moiety can be used to prevent the interaction of mCD163 with CD163-ligand, serving to stimulate an immune response. This is particularly useful for the therapeutic or prophylactic treatment of an individual with an insufficient immune response disorder, like cancer, infections and other disease characterized by insufficient immune responses.

On the other hand a CD163-ligand molecule of the invention, a CD163-ligand molecule of the invention coupled to a moiety (e.g., the constant region of an immunoglobulin (e.g. sCD163-ligand-Fc)), an agonist I of a CD163-ligand molecule of the invention, an agonist I of a CD163-ligand molecule of the invention coupled to a moiety, an agonist II of CD163, and/or an agonist II of CD163 coupled to a moiety can serve to suppress an immune response. This is particularly useful for the therapeutic or prophylactic treatment of an individual with an immune response disorder, like inflammatory diseases, autoimmune diseases, transplantation, allergy, asthma, etc.

In another aspect, the invention provides use of a CD163-ligand molecule according to the invention, an antagonist I of a CD163-ligand molecule according to the invention, an agonist I of a CD163-ligand molecule according to the invention, an isolated CD163 according to the invention, an antagonist II of an isolated CD163 according to the invention, an agonist II of an isolated CD163 according to the invention, a cell according to the invention, and/or a gene delivery vehicle according to the invention in the preparation of a pharmaceutical composition, wherein the disease comprises an autoimmune disease (e.g., rheumatoid arthritis, etc.), inflammatory disease (e.g., inflammatory bowel disease, etc.) allergy, asthma, cancer, infectious diseases (e.g., sepsis, etc.), transplantation-related diseases, host versus graft-related diseases, cardiovascular disease, and/or neurological diseases. It is understood that all diseases associated with increased serum sCD163 levels are also included.

The invention provides a method of treatment of an immune-related disease comprising a CD163-ligand molecule according to the invention, an antagonist I of a CD163-ligand molecule according to the invention, an agonist I of a CD163-ligand molecule according to the invention, an isolated CD163 according to the invention, an antagonist II of an isolated CD163 according to the invention, an agonist II of an isolated CD163 according to the invention, a cell according to the invention, and/or a gene delivery vehicle according to the invention with a carrier to a suitable recipient. A suitable recipient, for example, is a mammal, preferably human.

The invention is further explained with the aid of the following illustrative Examples.

### EXAMPLES

### Example 1

### Identification of Putative CD163-Ligand Molecules Using (s)CD163.

Generation of sCD163 from Human Monocytes

To isolate sCD163, peripheral blood mononuclear cells (PBMC) were isolated from a buffy coat by density centrifugation on lymphoprep (Nycomed, Oslo, Norway), followed by three washes. This was followed by gradient centrifugation on Percoll (Pharmacia, Uppsala, Sweden) to isolate monocytes. The monocytes were resuspended in RPMI (BioWhittaker, Walkersville, Md.) supplemented with 5% human pool serum (BioWhittaker, Walkersville, Md.) and gentamycin and cultured for 72 hours in the presence of 200 nM dexamethasone (Sigma, St. Louis, Mo.) to enhance CD163 expression. Upon induction of cell surface CD163, shedding of CD163 was induced by washing the cells in serum-free PBS, resuspending them in PBS in the presence of 50 nM PMA (Sigma, St. Louis, Mo.). After a one-hour incubation at 37° C., supernatants were collected to measure sCD163 levels in SDS-PAGE and western blotting analysis using commercially available anti-CD163 mAbs. In addition, removal of CD163 from the cell surface was assessed by FACS analysis using commercially available anti-CD163 mAbs. sCD163 was diluted five-fold with 20 mM sodium phosphate buffer (pH 7.0), and sCD163 was purified from these supernatants by conventional column chromatography methods (SP sepharose followed by Q sepharose column).

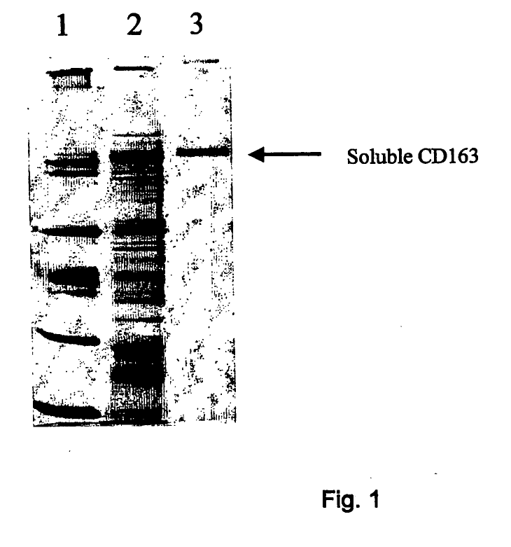

An SP Sepharose-column (SP Sepharose fast flow, Amersham Biosciences, Uppsala, Sweden) was equilibrated with 20 mM sodium phosphate buffer (pH 7.0), the sample was loaded at 0.5 ml/minute, and the flow-through that contained the sCD163 was collected in several fractions. The sCD163-containing fractions were pooled and loaded at 0.5 ml/minute onto a Q Sepharose-column (Q Sepharose fast flow Amersham Biosciences, Uppsala, Sweden) that was previously equilibrated with 20 mM sodium phosphate buffer (pH 7.0). sCD163 was eluted from the column by a 0-100% gradient of 20 mM sodium phosphate buffer/1 M NaCl (pH 7.0) (FIG. 1).

Biotinylation of sCD163

sCD163-containing fractions were collected from sCD163 purified as described above and fractions containing sCD163, as evidenced by SDS-PAGE analysis and western blotting, were pooled and dialyzed against 0.1 M NaHCO₃ (pH 8.6). N-hydroxysuccinimide-biotin (Perbio Science, Etten-Leur, the Netherlands) was dissolved in DMSO and was added to the CD163 in a 1:10 w/w ratio. The mixture was incubated at room temperature for four hours, and dialyzed extensively against PBS. BSA was added to a final concentration of 0.2% and NaN₃ was added to a final concentration of 0.1%. Biotin labeling of sCD163 was confirmed by western blotting using streptavidin-HRP (Jackson Immunoresearch Laboratories, West Grove, Pa.).

SCD163-Biotin Binds to Apoptotic Cells

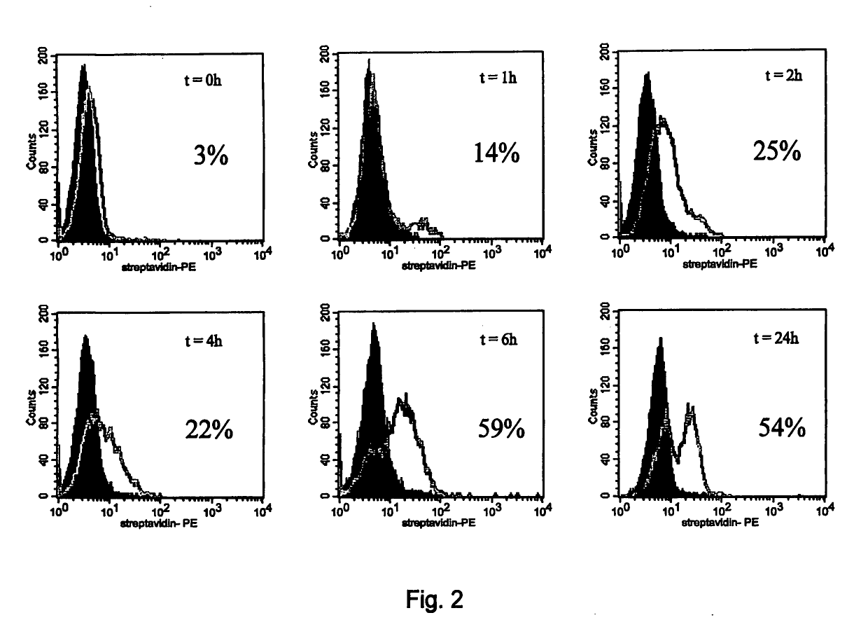

To study binding of sCD163 to a putative ligand, initial experiments were performed on peripheral blood mononuclear cells. PBMC isolated as described above were incubated 15-30 minutes at 37° C. in PBS 0.1% BSA in the presence or absence of sCD163-biotin (1:5-1:10 dilution), followed by washing and incubation in the presence of streptavidin-PE (1:100, Jackson Immunoresearch Laboratories, West Grove, Pa.). Cells were washed and analyzed by FACS analysis (FACScan, Becton & Dickinson, Erembodegem, Belgium). This showed that sCD163-biotin bound to a minor fraction of lymphocytes present in the PBMC. Surprisingly, the sCD163-biotin (but not streptavidin-PE by itself) bound to cells that had a forward scatter-side scatter profile resembling (pre)apoptotic cells. Similar results were found by using the human T-cell line Jurkat E6-1 (ATCC, Rockville, Md.). To obtain more apoptotic Jurkat E6-1 cells, apoptosis was induced by treating the cells with anti-CD95 antibody (clone CH11; 20 ng/ml, Campro Scientific, Veenendaal, the Netherlands). As can be seen in FIG. 2, the number of early apoptotic cells increased in the first six hours of incubation. Simultaneously, sCD163-biotin binding increased to reach >50% of these early apoptotic Jurkat E6-1 cells.

Identification of CD163-Ligand Molecules on Apoptotic Jurkat E6-1 T-Cells

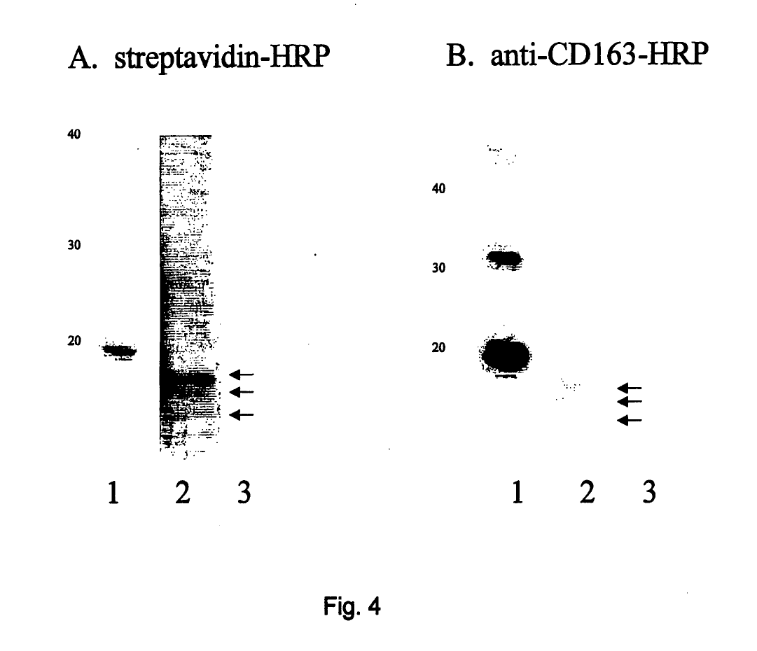

When these cells were lysed in the non-ionic detergent Nonidet P40 (NP40), three distinct bands at approximately 10 to 20 kDa were induced, all three of which bound sCD163 (detected with anti-CD163), as well as CD163-biotin (detected with streptavidin-HRP) in western blots (FIGS. 4A and 4B).

Mass Analyses of CD163-Ligand Proteins on Apoptotic Jurkat E6-1 T-Cells

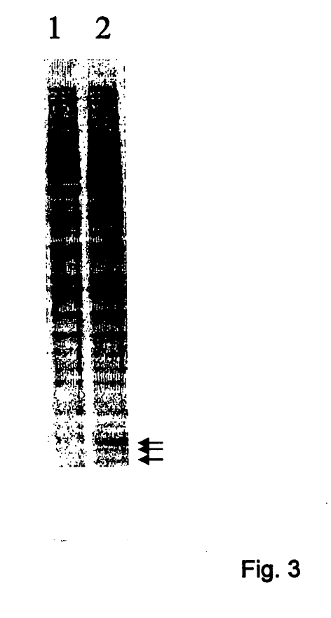

The three 10-20 kDa protein bands of interest (see FIG. 3) were cut from the gel after Coomassie staining. For MALDI analysis, protein-containing gel slices were S-alkylated with iodoacetamide, digested with trypsin (Roche Molecular Biochemicals, sequencing grade), and extracted according to Shevchenko et al. (Shevchenko et al. 1996). Only peptides eluted with 20 mM NH₄HCO₃ were used in the analysis. After drying in a vacuum centrifuge, peptides were dissolved in 10 μl of a solution containing 1% formic acid and 60% acetonitrile. Eluted peptides were mixed 1:1 (vlv) with a solution containing 52 mM α-cyano-4-hydroxycinnamic acid (Sigma-Aldrich Chemie BV) in 49% ethanol/49% acetonitril/2% TFA and 1 mM Ammoniumacetate (0.5 μl with 0.5 μl). Prior to dissolving, the α-cyano-4-hydroxycinnamic acid was washed briefly with acetone. The mixture was spotted on target and allowed to dry at room temperature. Reflectron MALDI-TOF spectra were acquired on a Micromass M@LDI (Wythenshawe, UK). The resulting peptide spectra were used to search several Non-Redundant Protein Databases. For further MSMS analysis, a few μl of several peptide solutions was concentrated on a C18 ZipTip (Millipore), eluted in 5 microliter 60% acetonitrile/1% HCOOH and analyzed on a Micromass Q-TOF mass spectrometer. Low-energy collision-induced dissociation (CID) experiments were performed using argon as a collision gas. Homology searches with fragmented peptides were performed using the BLASTx program with default settings and dbEST database (at http://www.ncbi.nlm.nih.gov:80/blast/Blast.cgi and http://www.ncbi.nlm.nih.gov/dbEST/) and with the MASCOT routine (Matrixscience).

Results: database search with MASCOT routine (Matrixscience)

Sample T (top band)=datafile EXT0878: Histone H2B, 3 MSMS spectra hits.

40 S ribosomal protein, 1 hit and Histone H2A, 1 hit.

Sample M (middle band)=datafile EXT0879: Histone H2A, 2 hits.

Sample B (band below)=datafile EXT0880: Profilin 1. 3 hits and Histone H4, 2 hits.

Taken together, the data presented above demonstrates that the three bands consist mostly of human H2B, H2A and H4.

In conclusion, the experiments described here show that sCD163 is capable to bind to ligands present on the membranes of early apoptotic cells other than haptoglobin-hemoglobin complexes. The expression of these ligands may be very important for the removal of early apoptotic cells from the body, thus preventing the presence of necrotic cells and tissue damage leading to inflammation.

### Example 2

### cDNA Cloning, Expression and Purification of Recombinant CD163-Ligand

Ligand molecules to human CD163, such as histones 2A (H2A, H2B, and H4), can be cloned using PCR primers based on known nucleotide sequences, or (partial) amino acid sequences of CD163-ligand molecules as described in Example 1. PCR reactions can be performed on mRNA derived from T-cells with known CD163-ligand expression, cloned into a plasmid vector, sequenced, amplified and expressed in several expression systems. Cloning and expression is carried out using standard techniques known to one of skill in the art. CD163-ligand molecules are expressed and purified by affinity purification using sCD163 or anti-CD163-ligand (e.g., anti-H2A, -H2B or -H4 mAb) Mabs or by conventional chromatography methods.

### Example 3

### Generation of a Soluble CD163-Ligand and a CD163-Ligand-Fc Fusion Protein

CD163-ligand and/or fractions thereof, and/or a sCD163-ligand-Fc fusion protein, can be generated to be used as a therapeutic molecule. CD163-ligand or truncated versions thereof comprising the CD163-binding site are generated and expressed as described in Example 1. From these constructs, a highly soluble CD163-ligand (sCD163-ligand) that can be expressed at high levels is isolated. Further, the sCD163-ligand is expressed as fusion protein containing the extracellular part of the membrane molecules fused to the constant region of a human immunoglobulin, for example, of IgG4. Other isotypes, such as IgG1, IgG2a, IgG2b, and/or IgG3 may also be used for this purpose. For the cloning of the human CH4/hinge-CH3 region, PCR primers are designed based on the sequence by Ellison et al. (Ellison et al. 1982). To allow successful linkage of this fragment to the extracellular domains (ED) of CD163-ligand, a small linker of six amino acids was introduced. In this small linker, the BamHI cloning site was incorporated to allow the in-frame fusion of the CD163-ligand ED. The human IgG4 Fc region is cloned from stimulated human B-cells. Total RNA is isolated and by RT-PCR, the Fc region is cloned and sequence verified. For expression, the Fc region is subcloned in the baculovirus-expression vector pVL1393. By DNA sequence analysis, the construct is checked. Using suitable primers starting from activated human T-cells, the CD163-ligand or a functional part thereof is amplified by RT-PCR (see Example 2). After sequence analysis, the correct clones are cloned using BamHI in the already mentioned pVL1393-expression vector containing human Fc. About 3×10⁶ Sf9 insect T-cells are plated in a 25 cm² culture flask and co-transfected with 2 mg CD163-ligand-Fc transfer vector together with 0.5 mg wild-type linearized baculovirus. After three days, the virus is re-amplified on fresh Sf9 cells, followed by several new rounds to obtain recombinant baculovirus stocks for expression. By Western blot analysis, expression of both proteins is confirmed.

### Example 4

### In vitro Immunomodulatory Effect of sCD163-Ligand and sCD163-Ligand-Fc

The immunomodulatory effect of sCD163-ligand and sCD163-ligand-Fc can be studied by performing a range of immunological assays. Mixed lymphocyte reactions (MLR), as well as antigen-specific T-cell proliferation assays against antigens as tetanus toxoid, candida albicans, or house dust mite are performed in the absence or presence of sCD163-ligand and/or sCD163-ligand-Fc. Such experiments can be used to demonstrate that sCD163-ligand and/or sCD163-ligand-Fc inhibit the proliferation of allo-specific (in the MLR) as well as antigen-specific T-cell proliferation. Further, modulation of stimulation of CD163-expressing monocytes and/or macrophages using CD163-ligand and/or CD163-ligand-Fc can be studied using human monocytes (e.g., cultured in the presence of glucocorticoids to induce CD163 expression). The CD163-expressing monocytes are incubated in the presence or absence of a monocyte/macrophage stimulus, such as LPS, in the presence or absence of (s)CD163-ligand and/or (s)CD163-ligand-Fc. After 24 hours, supernatants of these cultures are collected and the levels of cytokines produced (such as TNF-alpha, IL-1beta, IL-10 and others) are determined, showing that the monocytes/macrophages are induced by (s)CD163-ligand and/or (s)CD163-ligand-Fc to produce less pro-inflammatory cytokines, while enhancing the production of anti-inflammatory cytokines such as IL-10. Further, cells are collected and the expression of a range of cell surface molecules (e.g., CD14, CD40, MR, CD83, etc.) is studied using FACS analysis, to determine if the expression levels of a number of relevant T-cell surface markers is influenced as a result of treatment with (s)CD163-ligand and/or (s)CD163-ligand-Fc.

### Example 5

### In vivo Immunosuppressive Effect of (s)CD163-Ligand and/or (s)CD163-Ligand-Fc

The immunosuppressive activity of (s)CD163-ligand and/or (s)CD163-ligand-Fc can be tested in a range of animal models for inflammatory disease. For this, human (s)CD163-ligand, (s)CD163-ligand-Fc, murine (s)CD163-ligand, and/or (s)CD163-ligand-Fc may be used. For example, (s)CD163-ligand and/or (s)CD163-ligand-Fc can be tested in a mouse model for multiple sclerosis, namely acute Experimental Allergic Encephalomyelitis (EAE). EAE can be induced in SJL/J mice with a synthetic peptide which is encephalitogenic in SJL/J. When mice are treated with (s)CD163-ligand and/or (s)CD163-ligand-Fc, efficacy can be shown by decreased clinical symptoms of EAE, as well as by histological analysis. Similar experiments can be performed by those skilled in the art in animal models for arthritis, allergy, asthma, transplantation, inflammatory bowel disease, and other inflammatory diseases.

### Example 6

### In vitro Neutralization of Immunostimulatory Effect of sCD163 by sCD163-Ligand and CD163-Ligand-Fc

The immunostimulatory effect of soluble sCD163 can be demonstrated by MLR as well as by antigen-specific T-cell proliferation assays (see Example 19). Similar assays can be performed in the absence or presence of (s)CD163-ligand and/or (s)CD163-ligand-Fc. Thus, the immunostimulatory effect of sCD163 can be overcome by the addition of (s)CD163-ligand and/or (s)CD163-ligand-Fc to the cultures, most likely as a result of preventing the blocking of membrane-bound CD163-ligand by sCD163. This can be proven to be correct in FACS experiments, in which labeled sCD163 is added to CD163-ligand-expressing T-cells in the absence or presence of (s)CD163-ligand and/or (s)CD163-ligand-Fc.

### Example 7

### Treatment of Human Inflammatory Diseases with (s)CD163-Ligand and/or (s)CD163-Ligand-Fc

A variety and myriad number of autoimmune and inflammatory diseases are indications for treatment with (s)CD163-ligand and/or (s)CD163-ligand-Fc molecules of the invention. These include, but are not limited to, rheumatoid arthritis, diabetes, multiple sclerosis, systemic lupus erythematosus, psoriasis, autoimmune thyroiditis, allergy, asthma, inflammatory bowel disease, septic shock, transplant rejection, atherosclerosis, other cardiovascular diseases, and Alzheimer's disease.

### Example 8

### Generation of Antibodies to CD163-Ligand

Polyclonal antiserum against CD163-ligand (for example histone 2A, histone 2B and/or histone 4) is raised by immunization of rabbits with recombinant human CD163-ligand or fractions thereof (e.g., four injections/animal; 20-200 μg/injection). After the final booster, the animals are bled to determine the titer of the polyclonal antiserum. To obtain monoclonal antibodies, six to eight week old Balb/c mice are immunized with recombinant human CD163-ligand or fractions thereof (for example, four times with two week intervals with 10-100 μg/injection dissolved in Freunds complete adjuvans for the first injection and Freunds incomplete adjuvans for subsequent immunizations). Splenocytes are isolated and fused with a fusion cell line (Sp2/0 myeloma cells), followed by limiting dilution. Growing clones are screened using, for example, an enzyme-linked immunosorbant assay (ELISA). Therefore, 96-well plates are coated with CD163-ligand or with a control protein. The culture supernatant is added, followed by washing and addition of a labeled anti-mouse antibody for detection. After limited dilution cloning of CD163-ligand-specific antibody producing hybridomas, stable hybridomas are obtained. From each clone, cell supernatant are collected and by affinity chromatography using protein A sepharose columns (Pharmacia, Uppsala, Sweden), monoclonal antibodies are purified. Based on their blocking abilities and/or their functional activities, such antibodies can be subdivided into agonist I, antagonist I and/or non-blocking, non-agonist antibodies.

### Example 9

### In vitro Immunomodulatory Effect of Agonist I and Antagonist I Antibodies to CD163-Ligand

The immunomodulatory effect CD163-ligand-specific agonist I and/or antagonist I antibodies can be studied by performing a range of immunological assays. Mixed lymphocyte reactions (MLR), as well as antigen-specific T-cell proliferation assays against antigens as tetanus toxoid, candida albicans, or house dust mite are performed in the absence or presence of CD163-ligand-specific agonist I and/or antagonist I antibodies. Such experiments can be used to demonstrate that CD163-ligand-specific antagonist I antibodies stimulate the proliferation of allo-specific (in the MLR) as well as antigen-specific T-cell proliferation. Likewise, such experiments can be used to demonstrate that CD163-ligand-specific agonist I antibodies inhibit the proliferation of allo-specific (in the MLR) as well as antigen-specific T-cell proliferation. Further, supernatants of these cultures can be collected and the levels of cytokines produced (such as TNF-alpha, IL-1 beta, IL-10 and others) are determined, showing that the monocytes/macrophages are induced by the blocking effect of CD163-ligand-specific antagonist I antibodies to produce more pro-inflammatory cytokines like TNF-alpha, while inhibiting the production of anti-inflammatory cytokines such as IL-10. Further, cells are collected and the expression of a range of cell surface molecules (e.g., CD14, CD40, MR, CD83, etc.) is studied using FACS analysis, to determine if the expression levels of a number of relevant T-cell surface markers is influenced as a result of treatment with CD163-ligand-specific antagonist I antibodies. Also, inhibitory effects of agonist I antibodies on the activation of T-cells can be studied by activating T-cells with the cytokines as IL-2 and/or IL-15, by stimulation with mitogens as PMA, by stimulation with monoclonal antibodies (mAb) (for example, with a combination of anti-CD28 and anti-CD3 mAb), or in the absence of other stimuli. Activation of T-cells can be evaluated by measuring T-cell cytokines in the supernatants, T-cell proliferation and/or the expression of activation markers.

### Example 10

### In vivo Immunostimulatory Effect of Antagonist I CD163-Ligand-Specific Antibodies

The immunostimulatory effect of (mouse-specific and/or species cross-reactive) antagonist I CD163-ligand-specific antibodies can be tested in a range of animal models for cancer and/or infection known to those skilled in the art. One can study survival as well as histology to determine the immunostimulatory activity of antagonist I antibodies, to show that antagonist I antibodies enhance survival in cancer and/or infection through the stimulation of an adequate immune response to the tumor and/or infectious agent.

### Example 11

### Treatment of Cancer and/or Infection Using Antagonist I Antibodies to CD163-Ligand Molecules

A number of human cancers and/or infection by viruses, bacteria, fungi and/or parasites in which insufficient immune responses are raised to adequately remove the tumor cell and/or the infectious agent are indications for treatment with antagonist I CD163-ligand-specific antibodies.

### Example 12

### Effect of Agonist I CD163-Ligand-Specific Antibodies on Degranulation of Human Basophils

As CD163-ligand can be present on basophils and mast T-cells, the effect of CD163-ligand-specific agonist I antibodies on basophils that are triggered to degranulate, either as a result of cross-linking of the high-affinity receptor for IgE by anti IgE or by stimulation with complement factor C5a can be studied. It can be demonstrated that the up-regulation after activation as described above of two cell surface markers, namely CD203C (97A6) and CD63, is inhibited by agonist I CD163-ligand-specific antibodies, showing that agonist I antibodies prevent the degranulation of basophils.

### Example 13

### In vivo Immunosuppressive Effect of Agonist I CD163-Ligand-Specific Antibodies

The immunosuppressive activity of (mouse-specific and/or species cross-reactive) agonist I CD163-ligand-specific antibodies can be tested in a range of animal models for inflammatory disease. For example, agonist I CD163-ligand-specific antibodies can be tested in a mouse model for multiple sclerosis, namely acute Experimental Allergic Encephalomyelitis (EAE). EAE can be induced in SJL/J mice with a synthetic peptide which is encephalitogenic in SJL/J. When mice are treated with agonist I CD163-ligand-specific antibodies, efficacy can be shown by decreased clinical symptoms of EAE, as well as by histological analysis. Similar experiments can be performed by those skilled in the art in animal models for arthritis, allergy, asthma, transplantation, inflammatory bowel disease, and other inflammatory diseases.

### Example 14

### Treatment of Autoimmune and Inflammatory Diseases with Agonist I CD163-Ligand-Specific Antibodies

A variety and myriad number of autoimmune and inflammatory diseases are indications for treatment with agonist I CD163-ligand-specific antibodies of the invention. These include, but are not limited to, rheumatoid arthritis, diabetes, multiple sclerosis, systemic lupus erythematosus, psoriasis, autoimmune thyroiditis, allergy, asthma, inflammatory bowel disease, septic shock, transplant rejection, atherosclerosis, other cardiovascular diseases, and Alzheimer's disease.

### Example 15

### Identification of CD163-Ligand-Expressing Cells

Using the CD163-ligand-specific antibodies described in Example 8, the expression of CD163-ligand on a wide variety of cell types can be studied, for example, by fluorescence-activated cell sorter (FACS) analysis, and by immunohistochemistry. The expression of CD163-ligand can be shown on a number of cell types, including, but not limited to, lymphoid, myeloid and endothelial cells.

### Example 16

### Assay to Measure CD163-Ligand in Biological Fluids or Culture Supernatants

A method was developed in which an anti-CD163-ligand mAb or sCD163 is used to capture sCD163-ligand. After this step, the sCD163-ligand can be detected with another anti-CD163-ligand mAb or sCD163 that are labeled to allow detection. This assay can be used as a diagnostic procedure, or can be used to screen compound for their effect on removal of CD163-ligand from the cell membrane of CD163-ligand-expressing cells.

### Example 17

### Determination of C- and N-Terminus of sCD163

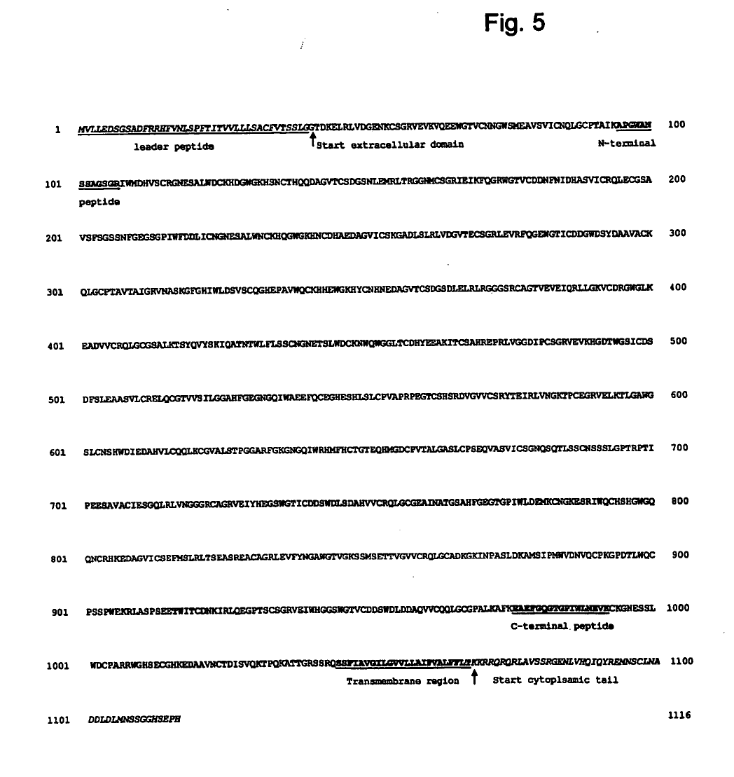

To determine the exact C- and N-terminus of sCD163 shed from monocytes peripheral blood mononuclear cells (PBMC) were isolated from sixteen buffy coats by density centrifugation on lymphoprep, followed by percoll density centrifugation. The monocytes were collected from the interphase, washed and suspended in RPMI, 5% human pool serum, and gentamycin as antibiotic. The monocytes were cultured for 48 hours in the presence of 200 nM dexamethasone. After 48 hours, the cells were washed and, resuspended in PBS (10⁷/ml). Shedding of membrane CD163 was induced by 50 nM PMA for 1.5 hours at 37° C. Cells were centrifuged and the supernatant containing sCD163 was removed and purified by ion exchange chromatography (IEX). For the determination of the C- and N-terminus, the sCD163 was subjected to electrophoresis on a denatured, reduced 7.5% SDS-PAGE gel, stained with coomassie blue, and the 130-155 kDa band (corresponding to the band of 110-130 kDa on the western blot made of a non-reduced SDS-PAGE) was excised for analysis of the N- and C-terminus by peptide mass fingerprinting. This analysis indicated that the 130-155 kDa (110-130 kDa under non-reducing circumstances) sCD163 was truncated at the C-terminus as well as at the N-terminus, resulting in a C-terminus of EAEFGQGTGPIWLNEVK (C-terminal residue: lysine) and an N-terminus of APGWANSSAGSGRIWMDHVSCR(N-terminal residue: alanine) (FIG. 5). It should be noted, however, that additional C- or N-terminal amino acids may be present but were not identified in the analysis.

### Example 18

### Generation of sCD163-Fc Fusion Proteins

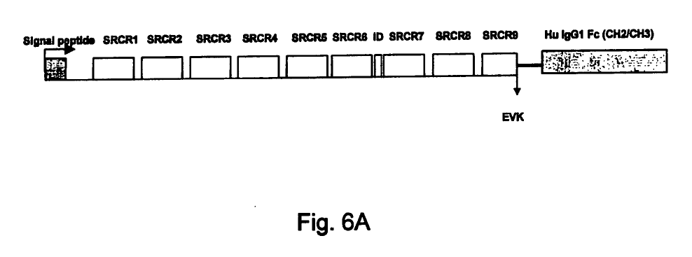

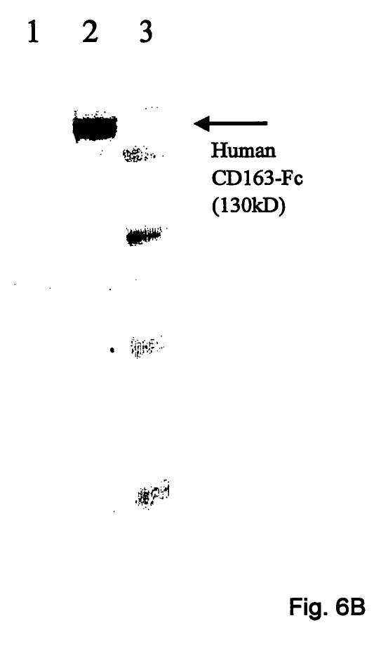

A cDNA-encoding human CD163 was cloned from dexamethasone-stimulated monocytes using PCR primers (SEQ 1 and SEQ 2) based on the literature. From this full-length clone, truncated variants were generated to generate an sCD163 (sCD163). The sCD163 was expressed in several expression systems and purified. A recombinant sCD163-Fc fusion protein was generated to be used as a therapeutic molecule (FIGS. 6A and 6B). For this molecule, the extracellular domain of human CD163 was cloned using PCR primers (SEQ 3 and SEQ 4). CD163 was expressed as fusion protein containing the extracellular part of the membrane molecule fused to the constant region of a human antibody, in this case IgG1. Other isotypes such as IgG4, IgG2a, IgG2b, and/or IgG3 may, however, also be used. For the cloning of a human Fc region, PCR primers (SEQ ID NO:5 and SEQ ID NO:6) were designed based on the published sequence. To allow successful linkage of this fragment to the extracellular domain of CD163, a small linker was introduced. In this small linker, the XbaI cloning site was incorporated to allow the in-frame fusion of the CD163 ED with the human IgG1 Fc part. The human IgG1 Fc region was cloned from stimulated human PBMC cells. Total RNA was isolated and by RT-PCR, the Fc region was cloned and sequence verified. Using the XbaI site, a clone encoding for the human CD163 Fc fusion protein was obtained (SEQ ID NO:11).

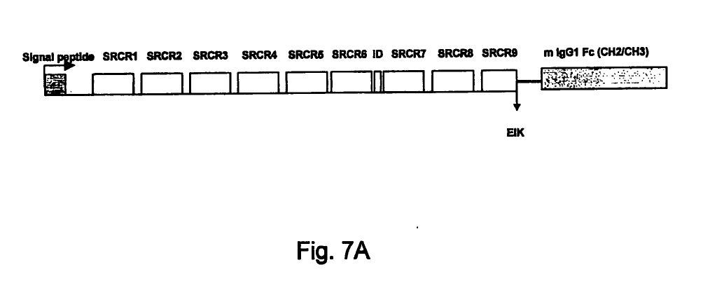

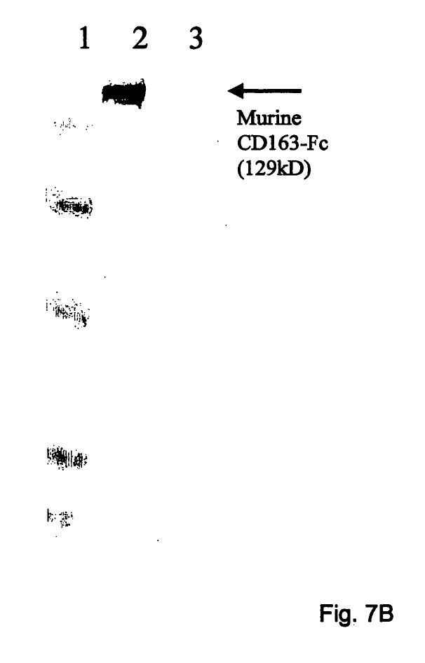

Similarly, a murine CD163 fusion protein was cloned (FIGS. 7A and 7B). cDNA encoding for the extracellular part of mCD163 was obtained by RT-PCR on spleen cells from dexamethasone-treated mice using PCR primers (SEQ ID NO:7 and SEQ ID NO:8). A murine IgG1 Fc region was cloned by RT-PCR using PCR primers (SEQ ID NO:9 and SEQ ID NO:10). As template, RNA was isolated from a murine hybridoma cell line expressing an IgG1 mAb. After sequence verification, a correct clone was obtained by fusing the mCD163 part with the mIgG1 part using BamHI (SEQ ID NO:12).

For expression, both the human and the murine fusion protein were subcloned in the baculovirus-expression vector pVL1393. About 3×10⁶ Sf9 insect T-cells were plated in a 25 cm² culture flask and co-transfected with 2 μg pVL1393 transfer vector containing the recombinant insert together with 0.5 μg wild-type linearized baculovirus. After three days, the virus was re-amplified on fresh Sf9 cells, followed by several new rounds to obtain recombinant baculovirus stocks for expression. By Western blot analysis, expression of both proteins was confirmed (FIGS. 6B and 7B).

### Example 19

### In vitro Immunostimulatory Effect of (s)CD163 and/or (s)CD163-Fc

The immunostimulatory effect of (s)CD163 and/or (s)CD163-Fc can be demonstrated by performing a range of immunological assays. Mixed lymphocyte reactions (MLR), as well as antigen-specific T-cell proliferation assays against antigens as tetanus toxoid, candida albicans, or house dust mite, are performed in the absence or presence of (s)CD163 and/or (s)CD163-Fc. Such experiments can be used to demonstrate that (s)CD163 and/or (s)CD163-Fc stimulate the proliferation of allo-specific (in the MLR) as well as antigen-specific T-cell proliferation. Further, supernatants of these cultures can be collected and the levels of cytokines produced (such as TNF-alpha, IL-1beta, IL-10 and others) are determined, showing that the monocytes/macrophages are induced by the blocking effect of (s)CD163 and/or (s)CD163-Fc to produce more pro-inflammatory cytokines like TNF-alpha, while inhibiting the production of anti-inflammatory cytokines such as IL-10. Further, cells are collected and the expression of a range of cell surface molecules (e.g., CD14, CD40, MR, CD83, etc.) is studied using FACS analysis, to determine if the expression levels of a number of relevant T-cell surface markers is influenced as a result of treatment with (s)CD163 and/or (s)CD163-Fc.

### Example 20

### In vivo Immunostimulatory Effect of (s)CD163 and/or (s)CD163-Fc

The immunostimulatory effect of (murine and/or human) (s)CD163 and/or (s)CD163-Fc can be tested in a range of animal models for cancer and/or infection known to those skilled in the art. One can study survival as well as histology to determine the immunostimulatory activity of (s)CD163 and/or (s)CD163-Fc, to show that (s)CD163 and/or (s)CD163-Fc enhance survival in cancer and/or infection through the stimulation of an adequate immune response to the tumor and/or infectious agent.

### Example 21

### Treatment of Cancer and/or Infection Using (s)CD163 and/or (s)CD163-Fc

A number of human cancers and/or infection by viruses, bacteria, fungi and/or parasites in which insufficient immune responses are raised to adequately remove the tumor cell and/or the infectious agent are indications for treatment with (s)CD163 and/or (s)CD163-Fc.

### Example 22

### Screening Assay to Identify Factors that Induce or Prevent CD163 Shedding from Cultured Monocytes/Macrophages

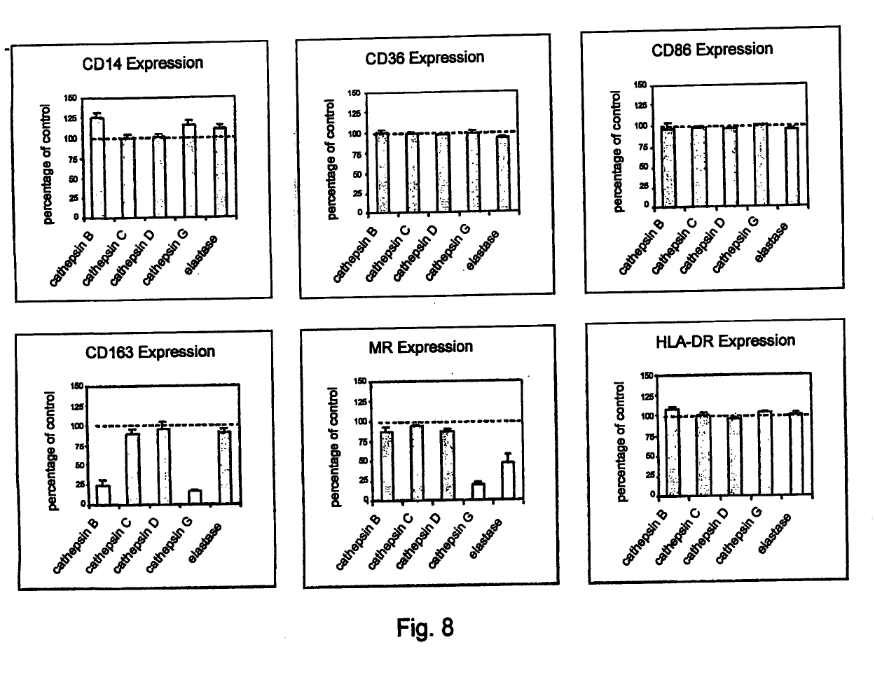

It is known that PMA, as well as LPS, can induce shedding of CD163 from the cell membranes. This process is inhibited by proteinase inhibitors, especially by metalloproteinase inhibitors as TIMP-3 or TAPI-0. To study the effect of other groups of isolated proteinases (aspartic, serine, and cysteine endoproteinases) on cell surface CD163 expression, monocytes were isolated from buffy coats. Monocytes were isolated from buffy coats as described in Example 1. The monocytes were resuspended in IMDM (BioWhittaker, Walkersville, Md.) supplemented with 5% human pool serum and gentamycin, and cultured for four days in the presence of dexamethasone (200 nM, Sigma, St. Louis, Mo.) to induce high levels of CD163 expression. The monocytes/macrophages were collected and incubated in the presence of PMA or without cathepsin B, cathepsin C, cathepsin D, cathepsin G, or elastase and incubated for one hour at 37° C. in PBS, 0.1% BSA. Expression of CD163, CD14, and HLA-DR was assessed by FACS analysis. As is shown in FIG. 8, cathepsin B and cathepsin G were able to remove CD163 from the cell membrane, but not other membrane proteins such as CD14, CD36, or HLA-DR. One can use this method, also extendible to test for the presence of sCD163 in supernatants using ELISA and/or Western blotting, to screen for additional compounds that remove CD163 from the cell surface (i.e., detecting factors that actively remove CD163 and/or compounds that down-regulate the expression of CD163).

### Example 23

### Generation of Antibodies to CD163

Polyclonal antiserum against (s)CD163 can be raised by immunization of rabbits with recombinant human (s)CD163 (e.g., four injections/animal; 20-200 μg/injection). After the final booster, the animals are bled to determine the titer of the polyclonal antiserum. To obtain monoclonal antibodies, six to eight-week old Balb/c mice are immunized with human sCD163 (for example, four times with two-week intervals with 10-100 μg/injection dissolved in Freunds complete adjuvans for the first injection and Freunds incomplete adjuvans for subsequent immunizations). Splenocytes are isolated and fused with a fusion cell line such as Sp2/0 myeloma cells, followed by limiting dilution. Growing clones are screened using, for example, an ELISA. Therefore, 96-well plates are coated with sCD163 or with a control protein. The culture supernatant is added, followed by washing and addition of a labeled anti-mouse antibody for detection. After limited dilution cloning of sCD163-specific antibody-producing hybridomas, stable hybridomas are obtained. From each clone, cell supernatant is collected and by affinity chromatography using protein A sepharose columns (Pharmacia, Uppsala, Sweden), monoclonal antibodies are purified. Monoclonal antibodies are further characterized by FACS analysis to determine binding of sCD163-specific mAbs to soluble, as well as to membrane-bound, CD163. Based on their blocking abilities and/or their functional activities, such mAb can be subdivided into agonist II, antagonist II and/or non-blocking, non-agonist mAb.

### Example 24

### Induction of Anti-Inflammatory Cytokines and Inhibition of Pro-Inflammatory Cytokines Using CD163-Specific Agonist II Antibodies In vitro

To study the possible role of cell surface CD163 in enhancing inflammatory responses, the effect of CD163 antibodies on LPS-induced activation of macrophages was studied. To this aim, monocytes were isolated from buffy coats and cultured in the presence or absence of dexamethasone as described in Example 1.

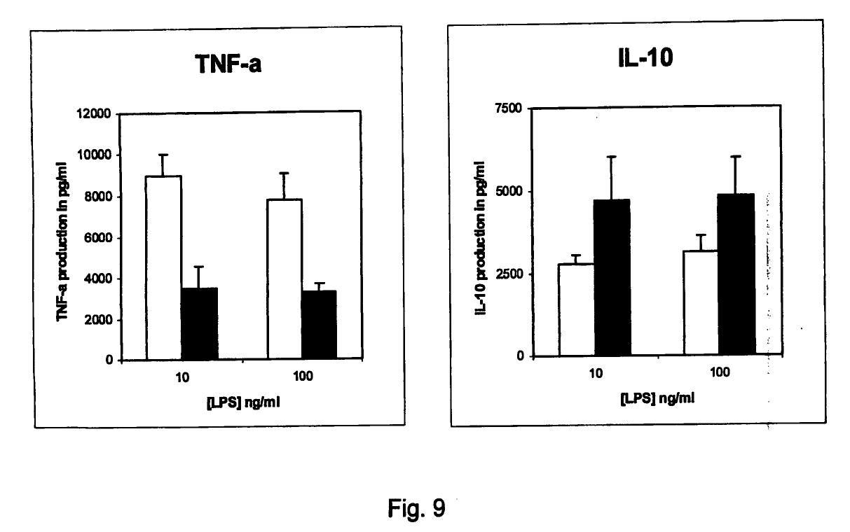

These CD163-expressing macrophages were stimulated with bacterial lipopolysaccharide (LPS, E. Coli strain 055:B5, Sigma, 100 ng/ml) in the absence or in the presence of anti-CD163 mAb RM3/1 (RDI Research Diagnostics, Flanders, N.J.) to mimic the interaction of CD163 with its putative ligand. After a 24-hour incubation, supernatants were collected and levels of pro- (TNF-alpha) and anti-inflammatory (IL-10) cytokines from these cultures were determined by enzyme-linked immunosorbent assay (ELISA) (CLB, Amsterdam, the Netherlands) after 24 hours incubation. Surprisingly, rather than increasing TNF-alpha production, addition of anti-CD163 antibodies dampened LPS-induced TNF-alpha responses from macrophages in two independent donors. In contrast, IL-10 levels were augmented under these conditions (FIG. 9). This was the case for dexamethasone-treated macrophages that had a very high CD163 expression, and also to a lesser extent for control macrophages, that expressed lower levels of CD163. However, CD163 has been shown to be enzymatically (metalloproteinase) cleaved from the cell membrane of dexamethasone-stimulated macrophages after exposure to LPS (Hintz et al. 2002). The CD163-positive macrophages described here were incubated with anti-CD163 mAb prior to LPS addition. After the incubation with LPS, however, CD163 was still present on the cell membrane, indicating that shedding of CD163 molecules induced by LPS is inhibited by CD163 ligation. Thus, CD163 ligation may have anti-inflammatory activity by preventing proteinase-induced tissue damage, as well as by shifting to a more anti-inflammatory profile of cytokine production.

### Example 25

### In vivo Immunosuppressive Effect of CD163-Specific Agonist II Antibodies

The immunosuppressive activity of CD163-specific agonist II antibodies can be tested in a range of animal models for inflammatory disease. For this, species cross-reactive human and/or mouse-specific CD163-specific agonist II antibodies may be used. For example, CD163-specific agonist II antibodies can be tested in a mouse model for multiple sclerosis, namely acute Experimental Allergic Encephalomyelitis (EAE). EAE can be induced in SJL/J mice with a synthetic peptide which is encephalitogenic in SJL/J. When mice are treated with CD163-specific agonist II antibodies, efficacy can be shown by decreased clinical symptoms of EAE as well as by histological analysis. Similar experiments can be performed by those skilled in the art in animal models for arthritis, allergy, asthma, transplantation, inflammatory bowel disease, and other inflammatory diseases.

### Example 26

### Treatment of Human Inflammatory Diseases with CD163-Specific Agonist II Antibodies

A variety and myriad number of autoimmune and inflammatory diseases are indications for treatment with CD163-specific agonist II antibodies of the invention. These include, but are not limited to, rheumatoid arthritis, diabetes, multiple sclerosis, systemic lupus erythematosus, psoriasis, autoimmune thyroiditis, allergy, asthma, inflammatory bowel disease, septic shock, transplant rejection, atherosclerosis, other cardiovascular diseases, and Alzheimer's disease.

### Example 27

### In vitro Immunostimulatory Effect of CD163-Specific Antagonist II Antibodies

The immunostimulatory effect of (s)CD163 and/or (s)CD163-Fc can be demonstrated by performing a range of immunological assays. Mixed lymphocyte reactions (MLR), as well as antigen-specific T-cell proliferation assays against antigens such as tetanus toxoid, candida albicans, or house dust mite are performed in the absence or presence of (s)CD163 and/or (s)CD163-Fc. Such experiments can be used to demonstrate that (s)CD163 and/or (s)CD163-Fc stimulate the proliferation of allo-specific (in the MLR) as well as antigen-specific T-cell proliferation. Further, supernatants of these cultures can be collected and the levels of cytokines produced (such as TNF-alpha, IL-1beta, IL-10 and others) are determined, showing that the monocytes/macrophages are induced by the blocking effect of (s)CD163 and/or (s)CD163-Fc to produce more pro-inflammatory cytokines like TNF-alpha, while inhibiting the production of anti-inflammatory cytokines such as IL-10. Further, cells are collected and the expression of a range of cell surface molecules (e.g., CD14, CD40, MR, CD83, etc.) is studied using FACS analysis, to determine if the expression levels of a number of relevant T-cell surface markers is influenced as a result of treatment with (s)CD163 and/or (s)CD163-Fc.

### Example 28

### In vivo Immunostimulatory Effect of CD163-Specific Antagonist II Antibodies

The immunostimulatory effect of (murine and/or human) (s)CD163 and/or (s)CD163-Fc can be tested in a range of animal models for cancer and/or infection known to those skilled in the art. One can study survival as well as histology to determine immunostimulatory activity of (s)CD163 and/or (s)CD163-Fc, to show that (s)CD163 and/or (s)CD163-Fc enhance survival in cancer and/or infection through the stimulation of an adequate immune response to the tumor and/or infectious agent.

### Example 29

### Treatment of Human Cancer and/or Infections with CD163-Specific Antagonist II Antibodies

A number of human cancers and/or infection by viruses, bacteria, fungi and/or parasites in which insufficient immune responses are raised to adequately remove the tumor cell and/or the infectious agent are indications for treatment with (s)CD163 and/or (s)CD163-Fc.

TABLE 1

SEQ ID NO:1

sense 5′-gcgc gatatctagaccaccatggtgctacttgaagactctggat-3′

SEQ ID NO:2

antisense 5′-gcgcgatatcgcggccgctcagtgtggctcagaatggcct-3′

SEQ ID NO:3

sense 5′-gcgcaagcttgtcgacgatatccaccatggtgctacttgaagactctgga-3′

SEQ ID NO:4

antisense 5′-gcgctctagacttcacttcattgagccatatcgg-3′

SEQ ID NO:5

sense 5′-gcgctctagaggatcccccgggctgcaggagcccaaatcttgtgacaaaact-3′

SEQ ID NO:6

antisense 5′-gcgcgcggccgctcatttacccggagacagggagaggct-3′

SEQ ID NO:7

sense 5′-gcgcaagcttgtcgacgatatccaccatgggtggacacagaatggttct-3′

SEQ ID NO:8

antisense 5′-gcgcggatcccttaatttcattgagccatatggg-3′

SEQ ID NO:9

sense 5′-gcgcggatcccccgggctgcaggtgcccagggattgtggttgtaa-3′

SEQ ID NO:10

antisense 5′-gcgcagatctgatatcgcggccgctcatttaccaggagagtgggaga-3′

SEQ ID NO:11

```
hCD163-hIgG1
M V L L E D S G S A D F R R H F V N L S
                10 20 30 40 50 60
atggtgctac ttgaagactc tggatctgct gacttcagaa gacattttgt caacctgagt
taccacgatg aacttctgag acctagacga ctgaagtctt ctgtaaaaca gttggactca
P F T I T V V L L L S A C F V T S S L G
                70 80 90 100 110 120
cccttcacca ttactgtggt cttacttctc agtgcctgtt ttgtcaccag ttctcttgga
gggaagtggt aatgacacca gaatgaagag tcacggacaa aacagtggtc aagagaacct
G T D K E L R L V D G E N K C S G R V E
              130 140 150 160 170 180
ggaacagaca aggagctgag gctagtggat ggtgaaaaca agtgtagcgg gagagtggaa
ccttgtctgt tcctcgactc cgatcaccta ccacttttgt tcacatcgcc ctctcacctt
V K V Q E E W G T V C N N G W S M E A V
              190 200 210 220 230 240
gtgaaagtcc aggaggagtg gggaacggtg tgtaataatg gctggagcat ggaagcggtc
cactttcagg tcctcctcac cccttgccac acattattac cgacctcgta ccttcgccag
S V I C N Q L G C P T A I K A P G W A N
              250 260 270 280 290 300
tctgtgattt gtaaccagct gggatgtcca actgctatca aagcccctgg atgggctaat
agacactaaa cattggtcga ccctacaggt tgacgatagt ttcggggacc tacccgatta
S S A G S G R I W M D H V S C R G N E S
              310 320 330 340 350 360
tccagtgcag gttctggacg catttggatg gatcatgttt cttgtcgtgg gaatgagtca
aggtcacgtc caagacctgc gtaaacctac ctagtacaaa gaacagcacc cttactcagt
A L W D C K H D G W G K H S N C T H Q Q
              370 380 390 400 410 420
gctctttggg attgcaaaca tgatggatgg ggaaagcata gtaactgtac tcaccaacaa
cgagaaaccc taacgtttgt actacctacc cctttcgtat cattgacatg agtggttgtt
D A G V T C S D G S N L E M R L T R G G
              430 440 450 460 470 480
gatgctggag tgacctgctc agatggatcc aatttggaaa tgaggctgac gcgtggaggg
ctacgacctc actggacgag tctacctagg ttaaaccttt actccgactg cgcacctccc
N M C S G R I E I K F Q G R W G T V C D
              490 500 510 520 530 540
aatatgtgtt ctggaagaat agagatcaaa ttccaaggac ggtggggaac agtgtgtgat
ttatacacaa gaccttctta tctctagttt aaggttcctg ccaccccttg tcacacacta
D N F N I D H A S V I C R Q L E C G S A
              550 560 570 580 590 600
gataacttca acatagatca tgcatctgtc atttgtagac aacttgaatg tggaagtgct
ctattgaagt tgtatctagt acgtagacag taaacatctg ttgaacttac accttcacga
V S F S G S S N F G E G S G P I W F D D
              610 620 630 640 650 660
gtcagtttct ctggttcatc taattttgga gaaggctctg gaccaatctg gtttgatgat
cagtcaaaga gaccaagtag attaaaacct cttccgagac ctggttagac caaactacta
L I C N G N E S A L W N C K H Q G W G K
              670 680 690 700 710 720
cttatatgca acggaaatga gtcagctctc tggaactgca aacatcaagg atggggaaag
gaatatacgt tgcctttact cagtcgagag accttgacgt ttgtagttcc tacccctttc
H N C D H A E D A G V I C S K G A D L S
              730 740 750 760 770 780
cataactgtg atcatgctga ggatgctgga gtgatttgct caaagggagc agatctgagc
gtattgacac tagtacgact cctacgacct cactaaacga gtttccctcg tctagactcg
L R L V D G V T E C S G R L E V R F Q G
              790 800 810 820 830 840
ctgagactgg tagatggagt cactgaatgt tcaggaagat tagaagtgag attccaagga
gactctgacc atctacctca gtgacttaca agtccttcta atcttcactc taaggttcct
E W G T I C D D G W D S Y D A A V A C K
              850 860 870 880 890 900
gaatggggga caatatgtga tgacggctgg gacagttacg atgctgctgt ggcatgcaag
cttaccccct gttatacact actgccgacc ctgtcaatgc tacgacgaca ccgtacgttc
Q L G C P T A V T A I G R V N A S K G F
              910 920 930 940 950 960
caactgggat gtccaactgc cgtcacagcc attggtcgag ttaacgccag taagggattt
gttgacccta caggttgacg gcagtgtcgg taaccagctc aattgcggtc attccctaaa
G H I W L D S V S C Q G H E P A V W Q C
              970 980 990 1000 1010 1020
ggacacatct ggcttgacag cgtttcttgc cagggacatg aacctgctgt ctggcaatgt
cctgtgtaga ccgaactgtc gcaaagaacg gtccctgtac ttggacgaca gaccgttaca
K H H E W G K H Y C N H N E D A G V T C
            1030 1040 1050 1060 1070 1080
aaacaccatg aatggggaaa gcattattgc aatcacaatg aagatgctgg cgtgacatgt
tttgtggtac ttaccccttt cgtaataacg ttagtgttac ttctacgacc gcactgtaca
S D G S D L E L R L R G G G S R C A G T
            1090 1100 1110 1120 1130 1140
tctgatggat cagatctgga gctaagactt agaggtggag gcagccgctg tgctgggaca
agactaccta gtctagacct cgattctgaa tctccacctc cgtcggcgac acgaccctgt
V E V E I Q R L L G K V C D R G W G L K
            1150 1160 1170 1180 1190 1200
gttgaggtgg agattcagag actgttaggg aaggtgtgtg acagaggctg gggactgaaa
caactccacc tctaagtctc tgacaatccc ttccacacac tgtctccgac ccctgacttt
E A D V V C R Q L G C G S A L K T S Y Q
            1210 1220 1230 1240 1250 1260
gaagctgatg tggtttgcag gcagctggga tgtggatctg cactcaaaac atcttatcaa
cttcgactac accaaacgtc cgtcgaccct acacctagac gtgagttttg tagaatagtt
V Y S K I Q A T N T W L F L S S C N G N
            1270 1280 1290 1300 1310 1320
gtgtactcca aaatccaggc aacaaacaca tggctgtttc taagtagctg taacggaaat
cacatgaggt tttaggtccg ttgtttgtgt accgacaaag attcatcgac attgccttta
E T S L W D C K N W Q W G G L T C D H Y
            1330 1340 1350 1360 1370 1380
gaaacttctc tttgggactg caagaactgg caatggggtg gacttacctg tgatcactat
ctttgaagag aaaccctgac gttcttgacc gttaccccac ctgaatggac actagtgata
E E A K I T C S A H R E P R L V G G D I
            1390 1400 1410 1420 1430 1440
gaagaagcca aaattacctg ctcagcccac agggaaccca gactggttgg aggggacatt
cttcttcggt tttaatggac gagtcgggtg tcccttgggt ctgaccaacc tcccctgtaa
P C S G R V E V K H G D T W G S I C D S
            1450 1460 1470 1480 1490 1500
ccctgttctg gacgtgttga agtgaagcat ggtgacacgt ggggctccat ctgtgattcg
gggacaagac ctgcacaact tcacttcgta ccactgtgca ccccgaggta gacactaagc
D F S L E A A S V L C R E L Q C G T V V
            1510 1520 1530 1540 1550 1560
gacttctctc tggaagctgc cagcgttcta tgcagggaat tacagtgtgg cacagttgtc
ctgaagagag accttcgacg gtcgcaagat acgtccctta atgtcacacc gtgtcaacag
S I L G G A H F G E G N G Q I W A E E F
            1570 1580 1590 1600 1610 1620
tctatcctgg gggqagctca ctttggagag ggaaatggac agatctgggc tgaagaattc
agataggacc cccctcgagt gaaacctctc cctttacctg tctagacccg acttcttaag
Q C E G H E S H L S L C P V A P R P E G
            1630 1640 1650 1660 1670 1680
cagtgtgagg gacatgagtc ccatctttca ctctgcccag tagcaccccg cccagaagga
gtcacactcc ctgtactcag ggtagaaagt gagacgggtc atcgtggggc gggtcttcct
T C S H S R D V G V V C S R Y T E I R L
            1690 1700 1710 1720 1730 1740
acttgtagcc acagcaggga tgttggagta gtctgctcaa gatacacaga aattcgcttg
tgaacatcgg tgtcgtccct acaacctcat cagacgagtt ctatgtgtct ttaagcgaac
V N G K T P C E G R V E L K T L G A W G
            1750 1760 1770 1780 1790 1800
gtgaatggca agaccccgtg tgagggcaga gtggagctca aaacgcttgg tgcctgggga
cacttaccgt tctggggcac actcccgtct cacctcgagt tttgcgaacc acggacccct
S L C N S H W D I E D A H V L C Q Q L K
            1810 1820 1830 1840 1850 1860
tccctctgta actctcactg ggacatagaa gatgcccatg ttctttgcca gcagcttaaa
agggagacat tgagagtgac cctgtatctt ctacgggtac aagaaacggt cgtcgaattt
C G V A L S T P G G A R F G K G N G Q I
            1870 1880 1890 1900 1910 1920
tgtggagttg ccctttctac cccaggagga gcacgttttg gaaaaggaaa tggtcagatc
acacctcaac gggaaagatg gggtcctcct cgtgcaaaac cttttccttt accagtctag
W R H M F H C T G T E Q H M G D C P V T
            1930 1940 1950 1960 1970 1980
tggaggcata tgtttcactg cactgggact gagcagcaca tgggagattg tcctgtaact
acctccgtat acaaagtgac gtgaccctga ctcgtcgtgt accctctaac aggacattga
A L G A S L C P S E Q V A S V I C S G N
            1990 2000 2010 2020 2030 2040
gctctaggtg cttcattatg tccttcagag caagtggcct ctgtaatctg ctcaggaaac
cgagatccac gaagtaatac aggaagtctc gttcaccgga gacattagac gagtcctttg
Q S Q T L S S C N S S S L G P T R P T I
            2050 2060 2070 2080 2090 2100
cagtcccaaa cactgtcctc gtgcaattca tcgtctttgg gcccaacaag gcctaccatt
gtcagggttt gtgacaggag cacgttaagt agcagaaacc cgggttgttc cggatggtaa
P E E S A V A C I E S G Q L R L V N G G
            2110 2120 2130 2140 2150 2160
ccagaagaaa gtgctgtggc ctgcatagag agtggtcaac ttcgcctggt aaatggagga
ggtcttcttt cacgacaccg gacgtatctc tcaccagttg aagcggacca tttacctcct
G R C A G R V E I Y H E G S W G T I C D
            2170 2180 2190 2200 2210 2220
ggtcgctgtg ctgggagagt agagatctat catgagggct cctggggcac catctgtgat
ccagcgacac gaccctctca tctctagata gtactcccga ggaccccgtg gtagacacta
D S W D L S D A H V V C R Q L G C G E A
            2230 2240 2250 2260 2270 2280
gacagctggg acctgagtga tgcccacgtg gtttgcagac agctgggctg tggagaggcc
ctgtcgaccc tggactcact acgggtgcac caaacgtctg tcgacccgac acctctccgg
I N A T G S A H F G E G T G P I W L D E
            2290 2300 2310 2320 2330 2340
attaatgcca ctggttctgc tcattttggg gaaggaacag ggcccatctg gctggatgag
taattacggt gaccaagacg agtaaaaccc cttccttgtc ccgggtagac cgacctactc
M K C N G K E S R I W Q C H S H G W G Q
            2350 2360 2370 2380 2390 2400
atgaaatgca atggaaaaga atcccgcatt tggcagtgcc attcacacgg ctgggggcag
tactttacgt taccttttct taqggcgtaa accgtcacgg taagtgtgcc gacccccgtc
Q N C R H K E D A G V I C S E F M S L R
            2410 2420 2430 2440 2450 2460
caaaattgca ggcacaagga ggatgcggga gttatctgct cagaattcat gtctctgaga
gttttaacgt ccgtgttcct cctacgccct caatagacga gtcttaagta cagagactct
L T S E A S R E A C A G R L E V F Y N G
            2470 2480 2490 2500 2510 2520
ctgaccagtg aagccagcag agaggcctgt gcagggcgtc tggaagtttt ttacaatgga
gactggtcac ttcggtcgtc tctccggaca cgtcccgcag accttcaaaa aatgttacct
A W G T V G K S S M S E T T V G V V C R
            2530 2540 2550 2560 2570 2580
gcttggggca ctgttggcaa gagtagcatg tctgaaacca ctgtgggtgt ggtgtgcagg
cgaaccccgt gacaaccgtt ctcatcgtac agactttggt gacacccaca ccacacgtcc
Q L G C A D K G K I N P A S L D K A M S
            2590 2600 2610 2620 2630 2640
cagctgggct gtgcagacaa agggaaaatc aaccctgcat ctttagacaa ggccatgtcc
gtcgacccga cacgtctgtt tcccttttag ttgggacgta gaaatctgtt ccggtacagg
I P M W V D N V Q C P K G P D T L W Q C
            2650 2660 2670 2680 2690 2700
attcccatgt gggtggacaa tgttcagtgt ccaaaaggac ctgacacgct gtggcagtgc
taagggtaca cccacctgtt acaagtcaca ggttttcctg gactgtgcga caccgtcacg
P S S P W E K R L A S P S E E T W I T C
            2710 2720 2730 2740 2750 2760
ccatcatctc catgggagaa gagactggcc agcccctcgg aggagacctg gatcacatgt
ggtagtagag gtaccctctt ctctgaccgg tcggggagcc tcctctggac ctagtgtaca
D N K I R L Q E G P T S C S G R V E I W
            2770 2780 2790 2800 2810 2820
gacaacaaga taagacttca ggaaggaccc acttcctgtt ctggacgtgt ggagatctgg
ctgttgttct attctgaagt ccttcctggg tgaaggacaa gacctgcaca cctctagacc
H G G S W G T V C D D S W D L D D A Q V
            2830 2840 2850 2860 2870 2880
catggaggtt cctgggggac agtgtgtgat gactcttggg acttggacga tgctcaggtg
gtacctccaa ggaccccctg tcacacacta ctgagaaccc tgaacctgct acgagtccac
V C Q Q L G C G P A L K A F K E A E F G
            2890 2900 2910 2920 2930 2940
gtgtgtcaac aacttggctg tggtccagct ttgaaagcat tcaaagaagc agagtttggt
cacacagttg ttgaaccgac accaggtcga aactttcgta agtttcttcg tctcaaacca
Q G T G P I W L N E V K S R G S P G L Q
            2950 2960 2970 2980 2990 3000
caggggactg gaccgatatg gctcaatgaa gtgaagtcta gaGGATCCCC CGGGCTGCAG
gtcccctgac ctggctatac cgagttactt cacttcagat ctCCTAGGGG GCCCGACGTC
E P K S C D K T H T C P P C P A P E L L
            3010 3020 3030 3040 3050 3060
GAGCCCAAAT CTTGTGACAA AACTCACACA TGCCCACCGT GCCCAGCACC TGAACTCCTG
CTCGGGTTTA GAACACTGTT TTGAGTGTGT ACGGGTGGCA CGGGTCGTGG ACTTGAGCAC
G G P S V F L F P P K P K D T L M I S R
            3070 3080 3090 3100 3110 3120
GGGGGACCGT CAGTCTTCCT CTTCCCCCCA AAACCCAAGG ACACCCTCAT GATCTCCCGG
CCCCCTGGCA GTCAGAAGGA GAAGGGGGGT TTTGGGTTCC TGTGGGAGTA CTAGAGGGCC
T P E V T C V V V D V S H E D P E V K F
            3130 3140 3150 3160 3170 3180
ACCCCTGAGG TCACATGCGT GGTGGTGGAC GTGAGCCACG AAGACCCTGA GGTCAAGTTC
TGGGGACTCC AGTGTACGCA CCACCACCTG CACTCGGTGC TTCTGGGACT CGAGTTCAAG
N W Y V D G V E V H N A K T K P R E E Q
            3190 3200 3210 3220 3230 3240
AACTGGTACG TGGACGGCGT GGAGGTGCAT AATGCCAAGA CAAAGCCGCG GGAGGAGCAG
TTGACCATGC ACCTGCCGCA CCTCCACGTA TTACGGTTCT GTTTCGGCGC CCTCCTCGTC
Y N S T Y R V V S V L T V L H Q D W L N
            3250 3260 3270 3280 3290 3300
TACAACAGCA CGTACCGGGT GGTCAGCGTC CTCACCGTCC TGCACCAGGA CTGGCTGAAT
ATGTTGTCGT GCATGGCCCA CCAGTCGCAG GAGTGGCAGG ACGTGGTCCT GACCGACTTA
G K E Y K C K V S N K A L P A P I E K T
            3310 3320 3330 3340 3350 3360
GGCAAGGAGT ACAAGTGCAA GGTCTCCAAC AAAGCCCTCC CAGCCCCCAT CGAGAAAACC
CCGTTCCTCA TGTTCACGTT CCAGAGGTTG TTTCGGGAGG GTCGGGGGTA GCTCTTTTGG
I S K A K G Q P R E P Q V Y T L P P S R
            3370 3380 3390 3400 3410 3420
ATCTCCAAAG CCAAAGGGCA GCCCCGAGAA CCACAGGTGT ACACCCTGCC CCCATCCCGG
TAGAGGTTTC GGTTTCCCGT CGGGGCTCTT GGTGTCCACA TGTGGGACGG GGGTAGGGCC
D E L T K N Q V S L T C L V K G F Y P S
            3430 3440 3450 3460 3470 3480
GATGAGCTGA CCAAGAACCA GGTCAGCCTG ACCTGCCTGG TCAAAGGCTT CTATCGCAGC
CTACTCGACT GGTTCTTGGT CCAGTCGGAC TGGACGGACC AGTTTCCGAA GATAGGGTCG
D I A V E W E S N G Q P E N N Y K T T P
            3490 3500 3510 3520 3530 3540
GACATCGCCG TGGAGTGGGA GAGCAATGGG CAGCCGGAGA ACAACTACAA GACCACGGCT
CTGTAGCGGC ACCTCACCCT CTCGTTACCC GTCGGCCTCT TGTTGATGTT CTGGTGCGGA
P V L D S D G S F F L Y S K L T V D K S
            3550 3560 3570 3580 3590 3600
CCCGTGCTGG ACTCCGACGG CTCCTTCTTC CTCTACAGCA AGCTCACCGT GGACAAGAGC
GGGCACGACC TGAGGCTGCC GAGGAAGAAG GAGATGTCGT TCGAGTGGCA CCTGTTCTCG
R W Q Q G N V F S C S V M H E A L H N H
            3610 3620 3630 3640 3650 3660
AGGTGGCAGC AGGGGAACGT CTTCTCATGC TCCGTGATGC ATGAGGCTCT GCACAACCAC
TCCACCGTCG TCCCCTTGCA GAAGAGTACG AGGCACTACG TACTCCGAGA CGTGTTGGTG
Y T Q K S L S L S P G K
            3670 3680 3690 3696
TACACGCAGA AGAGCCTCTC CCTGTCTCCG GGTAAA
ATGTGCGTCT TCTCGGAGAG GGACAGAGGC CCATTT
```

SEQ ID NO:12

```
mCD163-mIgG1
M G G H R M V L L G G A G S P G C K R F
                10 20 30 40 50 60
atgggtggac acagaatggt tcttcttgga ggtgctggat ctcctggttg taaaaggttt
tacccacctg tgtcttacca agaagaacct ccacgaccta gaggaccaac attttccaaa
V H L G F F V V A V S S L L S A S A V T
                70 80 90 100 110 120
gtccatctag gtttctttgt tgtggctgtg agctcacttc tcagtgcctc tgctgtcact
caggtagatc caaagaaaca acaccgacac tcgagtgaag agtcacggag acgacagtga
N A P G E M K K E L R L A G G E N N C S
              130 140 150 160 170 180
aacgctcctg gagaaatgaa gaaggaactg agactggcgg gtggtgaaaa caactgtagt
ttgcgaggac ctctttactt cttccttgac tctgaccgcc caccactttt gttgacatca
G R V E L K I H D K W G T V C S N G W S
              190 200 210 220 230 240
gggagagtgg aacttaagat ccatgacaag tggggcacag tgtgcagtaa cggctggagc
ccctctcacc ttgaattcta ggtactgttc accccgtgtc acacgtcatt gccgacctcg
M N E V S V V C Q Q L G C P T S I K A L
              250 260 270 280 290 300
atgaatgaag tgtccgtggt ttgccagcag ctgggatgcc caacttctat taaagccctt
tacttacttc acaggcacca aacggtcgtc gaccctacgg gttgaagata atttcgggaa
G W A N S S A G S G Y I W M D K V S C T
              310 320 330 340 350 360
ggatgggcta actccagcgc cggctctgga tatatctgga tggacaaagt ttcttgtaca
cctacccgat tgaggtcgcg gccgagacct atatagacct acctgtttca aagaacatgt
G N E S A L W D C K H D G W G K H N C T
              370 380 390 400 410 420
gggaatgagt cagctctttg ggactgcaaa catgatgggt ggggaaagca taactgtacc
cccttactca gtcgagaaac cctgacgttt gtactaccca cccctttcgt attgacatgg
H E K D A G V T C S D G S N L E M R L V
              430 440 450 460 470 480
catgaaaaag atgctggagt gacctgctca gatggatcta atttggagat gagactggtg
gtactttttc tacgacctca ctggacgagt ctacctagat taaacctcta ctctgaccac
N S A G H R C L G R V E I K F Q G K W G
              490 500 510 520 530 540
aacagtgcgg gccaccgatg cttaggaaga gtagaaataa agttccaggg aaagtggggg
ttgtcacgcc cggtggctac gaatccttct catctttatt tcaaggtccc tttcaccccc
T V C D D N F S K D H A S V I C K Q L G
              550 560 570 580 590 600
acggtgtgtg acgacaactt cagcaaagat cacgcttctg tgatttgtaa acagcttgga
tgccacacac tgctgttgaa gtcgtttcta gtgcgaagac actaaacatt tgtcgaacct
C G S A I S F S G S A K L G A G S G P I
              610 620 630 640 650 660
tgtggaagtg ccattagttt ctctggctca gctaaattgg gagctggttc tggaccaatc
acaccttcac ggtaatcaaa gagaccgagt cgatttaacc ctcgaccaag acctggttag
W L D D L A C N G N E S A L W D C K H R
              670 680 690 700 710 720
tggctcgatg acctggcatg caatggaaat gagtcagctc tctgggactg caaacaccgg
accgagctac tggaccgtac gttaccttta ctcagtcgag agaccctgac gtttgtggcc
G W G K H N C D R A E D V G V I C L E G
              730 740 750 760 770 780
ggatggggca agcataactg tgaccatgct gaggatgtcg gtgtgatttg cttagaggga
cctaccccgt tcgtattgac actggtacga ctcctacagc cacactaaac gaatctccct
A D L S L R L V D G V S R C S G R L E V
              790 800 810 820 830 840
gcagatctga gcctgagact agtggatgga gtgtccagat gttcaggaag attggaagtg
cgtctagact cggactctga tcacctacct cacaggtcta caagtccttc taaccttcac
R F Q G E W G T V C D D N W D L R D A S
              850 860 870 880 890 900
agattccaag gagaatgggg gaccgtgtgt gatgataact gggatctccg ggatgcttct
tctaaggttc ctcttacccc ctggcacaca ctactattga ccctagaggc cctacgaaga
V V C K Q L G C P T A I S A I G R V N A
              910 920 930 940 950 960
gtggtgtgca agcaactggg atgtccaact gccatcagtg ccattggtcg agttaatgcc
caccacacgt tcgttgaccc tacaggttga cggtagtcac ggtaaccagc tcaattacgg
S E G S G Q I W L D N I S C E G H E A T
              970 980 990 1000 1010 1020
agtgagggat ctggacagat ttggcttgac aacatttcat gcgaaggaca tgaggcaact
tcactcccta gacctgtcta aaccgaactg ttgtaaagta cgcttcctgt actccgttga
L W E C K H Q E W G K N Y C H H R E D A
            1030 1040 1050 1060 1070 1080
ctttgggagt gtaaacacca agagtgggga aagcattact gtcatcatag agaagacgct
gaaaccctca catttgtggt tctcacccct ttcgtaatga cagtagtatc tcttctgcga
G V T C S D G A D L E L R L V G G G S R
            1090 1100 1110 1120 1130 1140
ggtgtgacat gttctgatgg agcagatctg gaacttagac ttgtaggtgg aggcagtcgc
ccacactgta caagactacc tcgtctagac cttgaatctg aacatccacc tccgtcagcg
C A G I V E V E I Q K L T G K M C S R G
            1150 1160 1170 1180 1190 1200
tgtgctggca ttgtggaggt ggagattcag aagctgactg ggaagatgtg tagccgaggc
acacgaccgt aacacctcca cctctaagtc ttcgactgac ccttctacac atcggctccg
W T L A D A D V V C R Q L G C G S A L Q
            1210 1220 1230 1240 1250 1260
tggacactgg cagatgcgga tgtggtttgc agacagcttg gatgtggatc tgcgcttcaa
acctgtgacc gtctacgcct acaccaaacg tctgtcgaac ctacacctag acgcgaagtt
T Q A K I Y S K T G A T N T W L F P G S
            1270 1280 1290 1300 1310 1320
acccaggcta agatctactc taaaactggg gcaacaaata cgtggctctt tcctggatct
tgggtccgat tctagatgag attttgaccc cgttgtttat gcaccgagaa aggacctaga
C N G N E T T F W Q C K N W Q W G G L S
            1330 1340 1350 1360 1370 1380
tgtaatggaa atgaaactac tttttggcaa tgcaaaaact ggcagtgggg cggcctttcc
acattacctt tactttgatg aaaaaccgtt acgtttttga ccgtcacccc gccggaaagg
C D N F E E A K V T C S G H R E P R L V
            1390 1400 1410 1420 1430 1440
tgtgataatt tcgaagaagc caaagttacc tgctcaggcc acagggaacc cagactggtt
acactattaa agcttcttcg gtttcaatgg acgagtccgg tgtcccttgg gtctgaccaa
G G E I P C S G R V E V K H G D V W G S
            1450 1460 1470 1480 1490 1500
ggaggagaaa tcccatgctc tggtcgtgtg gaagtgaaac acggagacgt gtggggctcc
cctcctcttt agggtacgag accagcacac cttcactttg tgcctctgca caccccgagg
V C D F D L S L E A A S V V C R E L Q C
            1510 1520 1530 1540 1550 1560
gtctgtgatt ttgacttgtc tctggaagct gccagtgtgg tgtgcaggga attacaatgt
cagacactaa aactgaacag agaccttcga cggtcacacc acacgtccct taatgttaca
G T V V S I L G G A H F G E G S G Q I W
            1570 1580 1590 1600 1610 1620
ggaacagtcg tctctatcct agggggagca cattttggag aaggaagtgg acagatctgg
ccttgtcagc agagatagga tccccctcgt gtaaaacctc ttccttcacc tgtctagacc
G E E F Q C S G D E S H L S L C S V A P
            1630 1640 1650 1660 1670 1680
ggtgaagaat tCCAGTGTAG TGGGGATGAG TCCCATCTTT CACTATGCTC AGTGGCGCCC
ccacttctta aGGTCACATC ACCCCTACTC AGGGTAGAAA GTGATACGAG TCACCGCGGG
P L D R T C T H S R D V S V V C S R Y I
            1690 1700 1710 1720 1730 1740
CCGCTAGACA GAACTTGTAC CCACAGCAGG GATGTCAGCG TAGTCTGCTC AGGATACATA
GGCGATCTGT CTTGAACATG GGTGTCGTCC CTACAGTCGC ATCAGACGAG TGCTATGTAT
D I R L A G G E S S C E G R V E L K T L
            1750 1760 1770 1780 1790 1800
GATATTCGTC TGGCAGGCGG CGAGTCCTCC TGTGAGGGAA GAGTGGAGCT CAAGACACTC
CTATAAGCAG ACCGTCCGCC GCTCAGGAGG ACACTCCCTT CTCACGTCGA GTTCTGTGAG
G A W G P L C S S H W D M E D A H V L C
            1810 1820 1830 1840 1850 1860
GGAGCCTGGG GTCCCCTCTG CAGTTCTCAT TGGGACATGG AAGATGCTCA TGTCTTATGT
CCTCGGACGC CAGGGGAGAC GTCAAGAGTA ACCCTGTACC TTCTACGAGT ACAGAATACA
Q Q L K C G V A Q S J P E G A H F G K G
            1870 1880 1890 1900 1910 1920
CAGCAGCTGA AGTGTGGGGT TGCCCAATCT ATTCCAGAAG GAGCACATTT TGGGAAAGGA
GTCGTCGACT TCACACCCCA ACGGGTTAGA TAAGGTCTTC CTCGTGTAAA ACCCTTTCCT
A G Q V W S H M F H C T G T E E H I G D
            1930 1940 1950 1960 1970 1980
GCTGGTCAGG TCTGGAGTCA CATGTTCCAC TGCACTGGAA CTGAGGAACA TATAGGAGAT
CGACCAGTCC AGACCTCAGT GTACAAGGTG ACGTGACCTT GACTCCTTGT ATATCCTCTA
C L M T A L G A P T C S E G Q V A S V I
            1990 2000 2010 2020 2030 2040
TGCCTCATGA CTGCTCTGGG TGCGCCGACG TGTTCCGAAG GACAGGTGGC CTCTGTCATC
ACGGAGTACT GACGAGACCC ACGCGGCTGC ACAAGGCTTC CTGTCCACCG GAGACAGTAG
C S G N Q S Q T L L P C S S L S P V Q T
            2050 2060 2070 2080 2090 2100
TGCTCAGGAA ACCAATCCCA GACACTATTG CCATGTAGTT CATTGTCTCC AGTCCAAACA
ACGAGTCCTT TGGTTAGGGT CTGTGATAAC GGTACATCAA GTAACAGAGG TGAGGTTTGT
T S S T I P K E S E V P C I A S G Q L R
            2110 2120 2130 2140 2150 2160
ACAAGCTCTA CAATTCCAAA GGAGAGTGAA GTTCCCTGCA TAGCAAGTGG CCAGCTTCGC
TGTTCGAGAT GTTAAGGTTT CCTCTCACTT CAAGGGACGT ATCGTTCACC GGTCGAAGCG
L V G G G G R C A G R V E V Y H E G S W
            2170 2180 2190 2200 2210 2220
TTGGTAGGTG GAGGTGGTCG CTGCGCTGGA AGAGTGGAGG TCTACCACGA GGGCTCTTGG
AACCATCCAC CTCCACCAGC GACGCGACCT TCTCACCTCC AGATGGTGCT GCCGAGAACC
G T V C D D N W D M T D A N V V C K Q L
            2230 2240 2250 2260 2270 2280
GGCACCGTCT GTGATGACAA TTGGGATATG ACTGATGCCA ATGTGGTGTG CAAGCAGCTG
CCGTGGCAGA CACTACTGTT AAGCCTATAG TGACTACGGT TACACCACAC GTTCGTCGAC
D C G V A I N A T G S A Y F G E G A G A
            2290 2300 2310 2320 2330 2340
GACTGTGGCG TGGCAATTAA CGCCACTGGC TCTGCTTACT TCGGGGAAGG AGCAGGAGCT
CTGACACCGC ACCGTTAATT GCGGTGACCG AGACGAATGA AGCCCCTTCC TCGTCCTCGA
I W L D E V I C T G K E S H I W Q C H S
            2350 2360 2370 2380 2390 2400
ATCTGGCTAG ACGAAGTCAT CTGCACTGGG AAAGAGTCTC ATATTTGGCA GTGCCATTCA
TAGACCGATC TGCTTCAGTA GACGTGACCC TTTCTCAGAG TATAAACCGT CACGGTAAGT
H G W G R H N C R H K E D A G V I C S E
            2410 2420 2430 2440 2450 2460
CATGGCTGGG GACGCCATAA CTGCAGGCAC AAAGAAGATG CAGGTGTTAT CTGCTCCGAG
GTACCGACCC CTGCGGTATT GACGTCCGTG TTTCTTCTAC GTCCACAATA GACGAGGCTC
F M S L R L T N E A H K E N C T G R L E
            2470 2480 2490 2500 2510 2520
TTCATGTCTC TGAGGCTGAC CAACGAAGCC CACAAAGAAA ACTGCACAGG TCGCCTTGAA
AAGTACAGAG ACTCCGACTG GTTGCTTCGG GTGTTTCTTT TGACGTGTCC AGCGGAAGTT
V F Y N G T W G S I G S S N M S P T T V
            2530 2540 2550 2560 2570 2580
GTGTTTTACA ATGGTACATG GGGCAGTATT GGCAGTAGCA ATATGTCTCC AACCACTGTG
CACAAAATGT TACCATGTAC CCCGTCATAA CCGTCATCGT TATACAGAGG TTGGTGACAC
G V V C R Q L G C A D N G T V K P I P S
            2590 2600 2610 2620 2630 2640
GGGGTGGTGT GCCGTCAGCT GGGCTGTGCA GACAACGGGA CTGTGAAACC CATACCTTCA
CCGCACCACA CGGCAGTCGA CCCGACACGT CTGTTGCCCT GACACTTTGG GTATGGAAGT
D K T P S R P M W V D R V Q C P K G V D
            2650 2660 2670 2680 2690 2700
GACAAGACAC CATCCAGGCC CATGTGGGTA GATCGTGTGC AGTGTCCAAA AGGAGTTGAC
CTGTTCTGTG GTAGGTCCGG GTACACCCAT CTAGCACACG TCACAGGTTT TCCTCAACTG
T L W Q C P S S P W K Q R Q A S P S S Q
            2710 2720 2730 2740 2750 2760
ACTTTGTGGC AGTGCCCCTC GTCACCTTGG AAACAGAGAC AGGCCAGCCC CTCCTCCCAG
TGAAACACCG TCACGGGGAG CAGTGGAACC TTTGTCTCTG TCCGGTCGGG GAGGAGGGTC
E S W I I C D N K I R L Q E G H T D C S
            2770 2780 2790 2800 2810 2820
GAGTCCTGGA TCATCTGTGA CAACAAAATA AGACTCCAGG AAGGGCATAC AGACTGTTCT
CTCAGGACCT AGTAGACACT GTTGTTTTAT TCTGAGGTCC TTCCCGTATG TCTGACAAGA
G R V E I W H K G S W G T V C D D S W D
            2830 2840 2850 2860 2870 2880
GGACGTGTGG AGATCTGGCA CAAAGGTTCC TGGGGAACAG TGTGTGATGA CTCCTGGGAT
CCTGCACACC TCTAGACCGT GTTTCCAAGG ACCCCTTGTC ACACACTACT GAGGACCCTA
L N D A K V V C K Q L G C G Q A V K A L
            2890 2900 2910 2920 2930 2940
CTTAATGATG CTAAGGTTGT ATGTAAGCAG TTGGGCTGTG GCCAAGCTGT GAAGGCACTA
GAATTACTAC GATTCCAACA TACATTCGTC AACCCGACAC CGGTTCGACA CTTCCGTGAT
K E A A F G P G T G P I W L N E I K G S
            2950 2960 2970 2980 2990 3000
AAAGAAGCAG CATTTGGTCC AGGAACTGGG CCCATATGGC TCAATGAAAT TAAGGGATCC
TTTCTTCGTC GTAAACCAGG TCCTTGACCC GGGTATACCG AGTTACTTTA ATTCCCTAGG
P G L Q V P R D C G C K P C I C T V P E
            3010 3020 3030 3040 3050 3060
CCCGGGCTGC AGGTGCCCAG GGATTGTGGT TGTAAGCCTT GCATATGTAC AGTCCCAGAA
GGGCCCGACG TCCACGGGTC CCTAACACCA ACATTCGGAA CGTATACATG TCAGGGTCTT
V S S V F I F P P K P K D V L T I T L T
            3070 3080 3090 3100 3110 3120
GTATCATCTG TCTTCATCTT CCCCCCAAAG CCCAAGGATG TGCTCACCAT TACTCTGACT
CATAGTAGAC AGAAGTAGAA GGGGGGTTTC GGGTTCCTAC ACGAGTGGTA ATGAGACTGA
P K V T C V V V D I S K D D P E V Q F S
            3130 3140 3150 3160 3170 3180
CCTAAGGTCA CGTGTGTTGT GGTAGACATC AGCAAGGATG ATCCCGAGGT CCAGTTCAGC
GGATTCCAGT GCACACAACA CCATCTGTAG TCGTTCCTAC TAGGGCTCCA GGTCAAGTCG
W F V D D V E V H T A Q T Q P R E E Q F
            3190 3200 3210 3220 3230 3240
TGGTTTGTAG ATGATGTGGA GGTGCACACA GCTCAGACGC AACCCCGGGA GGAGCAGTTC
ACCAAACATC TACTACACCT CCACGTGTGT CGAGTCTGCG TTGGGGCCCT CCTCGTCAAG
N S T F R S V S E L P I M H Q D W L N G
            3250 3260 3270 3280 3290 3300
AACAGCACTT TCCGCTCAGT CAGTGAACTT CCCATCATGC ACCAGGACTG GCTCAATGGC
TTGTCGTGAA AGGCGAGTCA GTCACTTGAA GGGTAGTACG TGGTCCTGAC CGAGTTACCG
K E F K C R V N S A A F P A P I E K T I
            3310 3320 3330 3340 3350 3360
AAGGAGTTCA AATGCAGGGT CAACAGTGCA GCTTTCCCTG CCCCCATCGA GAAAACCATC
TTCCTCAAGT TTACGTCCCA GTTGTCACGT CGAAAGGGAC GGGGGTAGCT CTTTTGGTAG
S K T K G R P K A P Q V Y T I P P P K E
            3370 3380 3390 3400 3410 3420
TCCAAAACCA AAGGCAGACC GAAGGCTCCA CAGGTGTACA CCATTCCACC TCCCAAGGAG
AGGTTTTGGT TTCCGTCTGG CTTCCGAGGT GTCCACATGT GGTAAGGTGG AGGGTTCCTC
Q M A K D K V S L T C M I T D F F P E D
            3430 3440 3450 3460 3470 3480
CAGATGGCCA AGGATAAAGT CAGTCTGACC TGCATGATAA CAGACTTCTT CCCTGAAGAC
GTGTACCGGT TCCTATTTCA GTCAGACTGG ACGTACTATT GTCTGAAGAA GGGACTTCTG
I T V E W Q W N G Q P A E N Y K N T Q P
            3490 3500 3510 3520 3530 3540
ATTACTGTGG AGTGGCAGTG GAATGGGCAG CCAGCGGAGA ACTACAAGAA CACTCAGCCC
TAATGACACC TCACCGTCAC CTTACCCGTC GGTCGCCTCT TGATGTTCTT GTGAGTCGGG
I M D T D G S Y F V Y S K L N V Q K S N
            3550 3560 3570 3580 3590 3600
ATCATGGACA CAGATGGCTC TTACTTCGTC TACAGCAAGC TCAATGTGCA GAAGAGCAAC
TAGTACCTGT GTCTACCGAG AATGAAGCAG ATGTCGTTCG AGTTACACGT CTTCTCGTTG
W E A G N T F T C S V L H E G L H N H H
            3610 3620 3630 3640 3650 3660
TGGGAGGCAG GAAATACTTT CACCTGCTCT GTGTTACATG AGGGCCTGCA CAACCACCAT
ACCCTCCGTC CTTTATGAAA GTGGACGAGA CACAATGTAC TCCCGGACGT GTTGGTGGTA
T E K S L S H S P G K
            3670 3680 3690 3693
ACTGAGAAGA GCCTCTCCCA CTCTCCTGGT AAA
TGACTCTTCT CGGAGAGGGT GAGAGGACCA TTT
```

### REFERENCES

Buechler C., Ritter M., Orso E., Langmann T., Klucken J., Schmitz G. Regulation of scavenger receptor CD163 expression in human monocytes and macrophages by pro- and anti-inflammatory stimuli. J Leukoc. Biol. 2000; 67:97-103.
Byrne A., Reen D. J. Lipopolysaccharide induces rapid production of IL-10 by monocytes in the presence of apoptotic neutrophils. J. Immunol. 2002; 168:1968-77.
Droste A., Sorg C., Högger P. Shedding of CD163, a novel regulatory mechanism for a member of the scavenger receptor cysteine-rich family. Biochem. Biophys. Res. Commun. 1999; 256:110-3.
Ellison J. W., Berson B. J., Hood L. E. The nucleotide sequence of a human immunoglobulin C gammal gene. Nucleic Acids Res. 1982; 10:4071-9.
Fadok V. A., Bratton D. L., Konowal A., Freed P. W., Westcott J. Y., Henson P. M. Macrophages that have ingested apoptotic cells in vitro inhibit pro-inflammatory cytokine production through autocrine/paracrine mechanisms involving TGF-beta, PGE2, and PAF. J. Clin. Invest. 1998; 101:890-8.
Fadok V. A., Bratton D. L., Henson P. M. Phagocyte receptors for apoptotic cells: recognition, uptake, and consequences. J. Clin. Invest. 2001; 108:957-62.
Fadok V. A., Bratton D. L., Guthrie L., Henson P. M. Differential effects of apoptotic versus lysed cells on macrophage production of cytokines: role of proteases. J. Immunol. 2001; 166:6847-54.
Frings W., Dreier J., Sorg C. Only the soluble form of the scavenger receptor CD163 acts inhibitory on phorbol ester-activated T-lymphocytes, whereas membrane-bound protein has no effect. FEBS Lett. 2002; 526:93-6.
Guyre P. M., Morganelli P. M., Goulding N. J. Methods for detecting inflammation and inflammatory conditions. WO 01/73435.
Guyre P. M., Morganelli P. M., Goulding N. J. Methods for detecting inflammation and inflammatory conditions. U.S. 20010041177.
van den Heuvel M. M., Tensen C. P., van As J. H., Van den Berg T. K., Fluitsma D. M., Dijkstra C. D., Dopp E. A., Droste A., Van Gaalen F. A., Sorg C., Högger P., Beelen R. H. Regulation of CD163 on human macrophages: cross-linking of CD163 induces signaling and activation. J. Leukoc. Biol. 1999; 66:858-66.
Hintz K. A., Rassias A. J., Wardwell K., Moss M. L., Morganelli P. M., Pioli P. A., Givan A. L., Wallace P. K., Yeager M. P., Guyre P. M. Endotoxin induces rapid metalloproteinase-mediated shedding followed by up-regulation of the monocyte hemoglobin scavenger receptor CD163. J. Leukoc. Biol. 2002; 72:711-7.
Högger P., Dreier J., Droste A., Buck F., Sorg C. Identification of the integral membrane protein RM3/1 on human monocytes as a glucocorticoid-inducible member of the scavenger receptor cysteine-rich family (CD163). J. Immunol. 1998; 161:1883-90.
Högger P., Sorg C. SCD163 inhibits phorbol ester-induced lymphocyte proliferation. Biochem. Biophys. Res. Commun. 2001; 288:841-3.
Imler J. L., Hoffmann J. A. Toll receptors in innate immunity. Trends Cell Biol. 2001; 11:304-11.
Kopp E. B., Medzhitov R. The Toll-receptor family and control of innate immunity. Curr. Opin. Immunol. 1999; 11:13-8.
Kristiansen M., Graversen J. H., Jacobsen C., Sonne O., Hoffman H. J., Law S. K., Moestrup S. K. Identification of the hemoglobin scavenger receptor. Nature 2001; 409:198-201.
Law S. K., Micklem K. J., Shaw J. M., Zhang X. P., Dong Y., Willis A. C., Mason D. Y. A new macrophage differentiation antigen which is a member of the scavenger receptor superfamily. Eur. J. Immunol. 1993; 23:2320-5.
Lee T. S., Chau L. Y. Heme oxygenase-1 mediates the anti-inflammatory effect of interleukin-10 in mice. Nat. Med. 2002; 8:240-6.
Linehan S. A., Martinez-Pomares L., Gordon S. Mannose receptor and scavenger receptor: two macrophage pattern-recognition receptors with diverse functions in tissue homeostasis and host defense. Adv. Exp. Med. Biol. 2000; 479:1-14.
McDonald P. P., Fadok V. A., Bratton D., Henson P. M. Transcriptional and translational regulation of inflammatory mediator production by endogenous TGF-beta in macrophages that have ingested apoptotic cells. J. Immunol. 1999; 163:6164-72.
Matsushita N., Kashiwagi M., Wait R., Nagayoshi R., Nakamura M., Matsuda T., Högger P., Guyre P. M., Nagase H., Matsuyama T. Elevated levels of sCD163 in sera and fluids from rheumatoid arthritis patients and inhibition of the shedding of CD163 by TIMP-3. Clin. Exp. Immunol. 2002; 130:156-61.
Mills J. C., Stone N. L., Pittman R. N. Extranuclear apoptosis. The role of the cytoplasm in the execution phase. J. Cell. Biol. 1999; 146 703-8.
Möller H. J., Peterslund N. A., Graversen J. H., Moestrup S. K. Identification of the hemoglobin scavenger receptor/CD163 as a natural soluble protein in plasma. Blood. 2002; 99:378-80.
Möller H. J., Aerts H., Gronbaek H., Peterslund N. A., Hyltoft Petersen P., Hornung N., Rejnmark L., Jabbarpour E., Moestrup S. K. SCD163: a marker molecule for monocyte/macrophage activity in disease. Scand. J. Clin. Lab. Invest. Suppl. 2002; 237:29-33.
Morganelli P. M., Guyre P. M. IFN-gamma plus glucocorticoids stimulate the expression of a newly identified human mononuclear phagocyte-specific antigen. J. Immunol. 1988; 140:2296-304.
Morganelli P. M., Guyre P. M. Monoclonal antibodies specific for a human mononuclear phagocyte-specific antigen. U.S. Pat. No. 5,077,216.
Nauta A. J., Daha M. R., Kooten C., Roos A. Recognition and clearance of apoptotic cells: a role for complement and pentraxins. Trends Immunol. 2003; 24:148-54.
Otterbein L., Sylvester S. L., Choi A. M. Hemoglobin provides protection against lethal endotoxemia in rats: the role of heme oxygenase-1. Am. J. Respir. Cell. Mol. Biol. 1995; 13:595-601.
Pulford K., Micklem K., McCarthy S., Cordell J., Jones M., Mason D. Y. A monocyte/macrophage antigen recognized by the four antibodies GHI/61, Ber-MAC3, Ki-M8 and SM4. Immunology 1992; 75:588-95.
Re F., Strominger J. L. Toll-like receptor 2 (TLR2) and TLR4 differentially activate human dendritic cells. J. Biol. Chem. 2001; 276:37692-9.
Ritter M., Buechler C., Langmann T., Schmitz G. Genomic organization and chromosomal localization of the human CD163 (M130) gene: a member of the scavenger receptor cysteine-rich superfamily. Biochem. Biophys. Res. Commun. 1999; 260:466-74.
Reiter I., Krammer B., Schwamberger G. Cutting edge: differential effect of apoptotic versus necrotic tumor cells on macrophage antitumor activities. J. Immunol. 1999; 15; 163:1730-2.
Ritter M., Buechler C., Langmann T. and Schmitz G. Genomic organization and chromosomal localization of the human CD163 (M130) gene: a member of the scavenger receptor cysteine-rich Superfamily. Biochem. Biophys. Res. Commun. 1999; 260:466-474.
Ritter M., Buechler C., Kapinsky M., Schmitz G. Interaction of CD163 with the regulatory subunit of casein kinase II (CKII) and dependence of CD163 signaling on CKII and protein kinase C. Eur. J. Immunol. 2001; 31:999-1009.
Savill J., Dransfield I., Gregory C., Haslett C. A blast from the past: clearance of apoptotic cells regulates immune responses. Nat. Rev. Immunol. 2002; 2:965-75.
Schaer D. J., Boretti F. S., Hongegger A., Poehler D., Linnscheid P., Staege H., Muller C., Schoedon G., Schaffner A. Molecular cloning and characterization of the mouse CD163 homologue, a highly glucocorticoid-inducible member of the scavenger receptor cysteine-rich family. Immunogenetics 2001; 53:170-7.
Schaer D. J., Boretti F. S., Schoedon G., Schaffner A. Induction of the CD163-dependent hemoglobin uptake by macrophages as a novel anti-inflammatory action of glucocorticoids. Br. J. Haematol. 2002; 119:239-43.
Schaer D. J., Schoedon G., Schaffner A. Assignment of the CD163 antigen (CD163) to mouse chromosome 6 band F2 by radiation hybrid mapping. Cytogenet. Genome Res. 2002; 98:231B.
Shevchenko A., Wilm M., Vorm O., Mann M. Mass spectrometric sequencing of proteins from silver-stained polyacryl-amide gels. Anal. Chem. 1996; 68:850-858.
Sulahian T. H., Högger P., Wahner A. E., Wardwell K., Goulding N. J., Sorg C., Droste A., Stehling M., Wallace P. K., Morganelli P. M., Guyre P. M. Human monocytes express CD163, which is up-regulated by IL-10 and identical to p155. Cytokine 2000; 12:1312-21.
Sulahian T. H., Hintz K. A., Wardwell K., Guyre P. M. Development of an ELISA to measure sCD163 in biological fluids. J. Immunol. Methods 2001; 252:25-31.
Voll R. E., Herrmann M., Roth E. A., Stach C., Kalden J. R., Girkontaite I. Immunosuppressive effects of apoptotic cells. Nature 1997; 390:350-1.
Walter R. B., Bachli E. B., Schaer D. J., Ruegg R., Schoedon G. Expression of the hemoglobin scavenger receptor (CD163/HbSR) as immunophenotypic marker of monocytic lineage in acute myeloid leukemia. Blood 2003; 101:3755.
Willingham M. C. Cytochemical methods for the detection of apoptosis. J. Histochem. Cytochem. 1999; 47:1101-10.
Wu D., Ingram A., Lahti J. H., Mazza B., Grenet J., Kapoor A., Liu L., Kidd V. J., Tang D. Apoptotic release of histones from nucleosomes. J. Biol. Chem. 2002; 277:12001-8.
Zwadlo G., Voegeli R., Osthoff K. S., Sorg C. A mAb to a novel differentiation antigen on human macrophages associated with the down-regulatory phase of the inflammatory process. Adv. Exp. Cell. Biol. 1987; 55:295-304.
Zwadlo-Klarwasser G., Bent S., Haubeck H. D., Sorg C., Schmutzler W. Glucocorticoid-induced appearance of the macrophage subtype RM3/1 in peripheral blood of man. Int. Arch. Allergy Appl. Immunol. 1990; 91:175-80.

1. A method for identifying a molecule with immune-modulatory activity capable of interacting with CD163, said method comprising:
providing a candidate molecule;
providing the candidate molecule with a second molecule selected from the group consisting of CD163, a functional part of CD163, a derivative of CD163, and analogue of CD163, and/or combinations thereof, under suitable conditions for detecting interaction of the candidate molecule with CD163, and
detecting any interaction between said candidate molecule and said second molecule, and
determining whether the candidate molecule is capable of modulating an immune response.

2. The method according to claim 1, wherein the second molecule comprises an sCD163.

3. The method according to claim 1, wherein said second molecule is cell bound and/or soluble.

4. The method according to claim 1, wherein said candidate molecule comprises a proteinaceous portion.

5. A CD163-ligand molecule, said CD163-ligand molecule being isolated, recombinant, synthetic or any combination of isolated, recombinant, and/or synthetic, and said molecule identifiable by the method according to claim 1.

6. The CD163-ligand molecule of claim 5 wherein said CD163-ligand molecule comprises a histone or a functional fragment of a histone.

7. The CD163-ligand molecule of claim 5 coupled to a moiety.

8. The CD163-ligand molecule of claim 7, wherein said moiety comprises a constant region of an immunoglobulin.

9. A method of modulating an immune response in a subject, the method comprising using the CD163-ligand molecule of claim 5 to modulate the immune response in the subject.

10. The method according to claim 9, wherein said modulation involves suppressing the immune response.

11. An antagonist for CD163/CD163-ligand signaling.

12. The antagonist of claim 11, said antagonist selected from the group consisting of an antibody, a part of any antibody that antagonizes CD163/CD163-ligand signaling, an antibody derivative that antagonizes CD163/CD163-ligand signaling, and an antibody analogue that antagonizes CD163/CD163-ligand signaling.

13. The antagonist of claim 11, said antagonist coupled to a moiety.

14. The antagonist of claim 11, wherein said antagonist is capable of specifically binding a CD163-ligand molecule.

15. The antagonist of claim 14, wherein said antagonist comprises sCD163.

16. The antagonist of claim 11, wherein said antagonist specifically binds a CD163 molecule.

17. An agonist of CD163/CD163-ligand signaling.

18. The agonist of claim 17, wherein said agonist is selected from the group consisting of an antibody that agonizes CD163/CD163-ligand signaling, an antibody part that agonizes CD163/CD163-ligand signaling, an antibody derivative that agonizes CD163/CD163-ligand signaling, and an antibody analogue that agonizes CD163/CD163-ligand signaling.

19. The agonist of claim 17 coupled to a moiety.

20. The agonist of claim 17, wherein said agonist specifically binds a CD163-ligand molecule.

21. The agonist of claims 17, wherein said agonist specifically binds a CD163 molecule.

22. The agonist of claim 21, wherein said agonist comprises a soluble CD163 ligand.

23. The agonist of claim 22, wherein said soluble CD163 ligand is derived from a histone.

24. (canceled)

25. (canceled)

26. A CD163 or a functional part, derivative and/or analogue thereof, wherein said CD163 is isolated, recombinant, and/or synthetic.

27. The CD163 of claim 26, wherein said CD163 comprises an sCD163.

28. The CD163 of claim 26 coupled to a moiety.

29. The CD163 of claim 28, wherein said moiety comprises a constant region of an immunoglobulin.

30. An immunoglobulin capable of specifically binding a CD163-ligand.

31. The immunoglobulin of claim 30, wherein said CD163-ligand comprises a histone.

32. A method of detecting the presence of the CD163-ligand molecule of claim 5, in a sample, said method comprising:
contacting the sample with a binding molecule for the CD163-ligand molecule to form a complex; and
detecting for said complex in the sample.

33. The method according to claim 32, wherein said binding molecule comprises a compound selected from the group consisting of an antagonist for CD163/CD163-ligand signaling that is capable of specifically binding a CD163-ligand molecule, an antagonist for CD163/CD163-ligand signaling that comprises sCD163, and an agonist of CD163/CD163-ligand signaling that specifically binds a CD163-ligand molecule.

34. The method according to claim 32, wherein said binding molecule comprises membrane-bound CD163 and/or an sCD163.

35. A method for determining binding activity of the CD163-ligand molecule of claim 5 in a sample, said method comprising:
detecting the presence of a molecule by contacting the sample with a binding molecule for the CD163-ligand molecule to form a complex, and detecting for said complex in the sample, and
further determining the levels of binding molecule-molecule in the sample.

36. A nucleic acid sequence encoding the CD163-ligand molecule of claim 5.

37. A nucleic acid sequence encoding the antagonist of claim 15.

38. A nucleic acid sequence, said nucleic acid sequence being isolated and encoding the CD163 of claim 26.

39. A nucleic acid sequence, said nucleic acid sequence being isolated and encoding the immunoglobulin of claim 30.

40. A vector comprising the nucleic acid of claim 37.

41. A cell comprising the vector of claim 40.

42. A gene delivery vehicle comprising the vector of claim 40.

43. A process for producing
the CD163 ligand molecule of claim 5, or
in an organism, said process comprising:
inserting into the organism's genome at least one copy of a nucleic acid sequence encoding CD163-ligand molecule.

44. A method of modulating an immune response in a subject, said method comprising:
administering to the subject:
the immunoglobulin of claim 30
so as to modulate the subject's immune response.

45. The method according to claim 44, wherein said immune response includes an antigen-specific immune response.

46. A composition comprising:
the CD163-ligand molecule of claim 5
wherein said composition is suitable for administration to a subject in a pharmaceutically acceptable form or manner.

47. A method of augmenting or suppressing an immune response in a subject, the method comprising:
administering to the subject the composition of claim 46
so as to augment or suppress the subject's immune response.

48. A method of treating a disease in a subject, said method comprising:
administering to the subject a composition comprising
the gene delivery vehicle of claim 42,
so as to modulate the subject's immune response.

49. The method according to claim 48, wherein the disease is selected from the group consisting of an autoimmune disease, allergy, asthma, inflammatory disease, cancer, Alzheimer's disease, infectious disease, host versus graft-related disease, cardiovascular disease, neurological diseases, a disease associated with elevated serum sCD163 levels, and combinations of any thereof.

50. A method of modulating an immune response in a subject, the method comprising:
administering to the subject a composition comprising:
means for modulating CD163 in the subject, said means present in an amount sufficient to agonize or antagonize the immune response as desired, and, admixed therewith,
a pharmaceutically acceptable excipient.

51. The method according to claim 50, wherein said modulation of the subject's immune response comprises suppressing the subject's immune response.

52. The method according to claim 51 wherein the means for modulating CD163 in the subject is an agonist of CD163/CD163-ligand signaling.

53. The method according to claim 52, wherein said agonist specifically binds a CD163 molecule.

54. The method according to 53, wherein said agonist comprises a soluble CD163 ligand.

55. The method according to claim 54, wherein said soluble CD163 ligand is derived from a histone.

56. The method according to claim 50 wherein said modulation of the subject's immune response comprises augmenting the subject's immune response.

57. The method according to claim 56 wherein the means for modulating CD163 in the subject is an antagonist of CD163/CD163-ligand signaling.

58. The method according to claim 57 wherein the antagonist is sCD163.
